(19) United States
(12) Patent Application Publication
(10) Pub. No.: US 2026/0069439 A1
(43) Pub. Date: Mar. 12, 2026

(54) MULTI-MODAL FLOW MODULATING DEVICES FOR BLOOD VESSELS

(71) Applicant: Edwards Lifesciences Corporation, Irvine, CA (US)

(72) Inventors: Assaf Sharon, Tel Aviv (IL); Aviran Pitusi, Rosh Haayin (IL); Aviv Geva, Hod Hasharon (IL); Danny M. Garmahi, Hadera (IL); Michael Bukin, Pardes Hanna (IL); Yaeer E. Lev, Torrance, CA (US); Leonardo Paim Nicolau Da Costa, Tampa, FL (US)

(21) Appl. No.: 19/387,399

(22) Filed: Nov. 12, 2025

Related U.S. Application Data
(63) Continuation of application No. PCT/US2024/029613, filed on May 16, 2024.
(60) Provisional application No. 63/502,910, filed on May 17, 2023.

(51) Int. Cl. A61F 2/90; A61F 2/958; A61M 60/441; A61M 60/531; A61M 60/843

(52) U.S. Cl. CPC A61F 2/90; A61F 2/958; A61M 60/441; A61M 60/531; A61M 60/843

(57) ABSTRACT

A device may include a frame including an inflow end and an outflow end. The frame defines a lumen between the inflow end and the outflow end. A device may include a first expandable member coupled to at least one portion of an internal surface of the frame and including a first inflation port. The first expandable member defines a first volume configured to receive an inflation fluid therein through the first inflation port. The first expandable member is configured to be reversibly inflatable to a first plurality of inflation states to partially or fully occlude the lumen. The devices described herein may be used to alleviate symptoms from congestive heart failure or chronic kidney disease.

### CROSS-REFERENCE TO RELATED APPLICATION

This application is a continuation of International Application No. PCT/US2024/029613, filed May 16, 2024, which claims the benefit of U.S. Provisional Application Ser. No. 63/502,910, filed May 17, 2023, the contents of which are herein incorporated by reference.

### BACKGROUND

This disclosure relates generally to the field of medical devices and procedures, and more specifically to the field of blood flow management in blood vessels.

Chronic kidney disease (CKD) is a common comorbidity with many patients who suffer from chronic Heart Failure (HF). HF patients may also have elevated right atrium pressure, which may impair kidney function. In HF patients with elevated right atrium pressure, the kidneys may attempt to perform a diuresis process, but such a process may be difficult to perform efficiently due to the elevated pressure. For example, elevated right atrium pressure may hinder the ability of the kidneys to drive forward the flow of blood for accomplishing proper and efficient diuresis. Such unbalanced pressure coupled with the typical poor kidney efficiency of CKD patients may lead to an unending cycle of fluid overload for a person, which may result in an increase in congestion and heart failure admissions to the hospital.

### SUMMARY

In some aspects, the techniques described herein relate to a device for modulating blood flow in a vessel, the device including: a frame including an inflow end and an outflow end, the frame defining a lumen between the inflow end and the outflow end; and a first expandable member coupled to at least one portion of an internal surface of the frame and including a first inflation port, the first expandable member defining a first volume configured to receive an inflation fluid therein through the first inflation port, wherein the first expandable member is configured to be reversibly inflatable to a first plurality of inflation states to partially or fully occlude the lumen.

In some aspects, the techniques described herein relate to a system for modulating blood flow through a vessel, the system including: an inflation apparatus including a pump and a reservoir configured to contain an inflation fluid; a flow modulating device including: a frame including an inflow end and an outflow end, the frame defining a lumen including an internal surface between the inflow end and the outflow end, and one or more expandable members coupled to at least one portion of the internal surface of the frame and including an inflation port, the one or more expandable members each defining a volume configured to receive an inflation fluid therein through the inflation port; and an inflation line configured to fluidly connect the reservoir of the inflation apparatus to the volume of each of the one or more expandable members through each inflation port, wherein the one or more expandable members are each reversibly inflatable to a plurality of inflation states to partially or fully occlude the lumen of the frame.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various embodiments, with reference made to the accompanying drawings.

FIG. 1A illustrates an embodiment of a flow modulating device positioned in a vessel.

FIG. 1B illustrates a schematic of an embodiment of a system for modulating flow in a vessel.

FIGS. 2A-2C illustrate cross-sectional views of an embodiment of a flow modulating device in a plurality of inflation states.

FIGS. 2D-2E illustrate cross-sectional views of an embodiment of a flow modulating device having one or more stasis reduction flow paths.

FIGS. 3A-3C illustrate cross-sectional views of an embodiment of a flow modulating device in a plurality of inflation states.

FIG. 3D illustrates a cross-sectional view of an embodiment of a flow modulating device having one or more stasis reduction flow paths.

FIGS. 4A-4B illustrate cross-sectional views of an embodiment of a flow modulating device in a plurality of inflation states.

FIG. 4C illustrates a cross-sectional view of an embodiment of a flow modulating device having one or more stasis reduction flow paths.

FIGS. 5A-5C illustrate various embodiments of flow modulating devices having one or more expandable members coupled to various portions of an internal surface of a frame.

FIG. 6 illustrates a perspective view of an embodiment of a flow modulating device having a particularly shaped expandable member.

FIG. 7A illustrates a perspective view of an embodiment of a flow modulating device having a particularly shaped expandable member.

FIG. 7B illustrates a perspective view of an embodiment of a flow modulating device having a particularly shaped expandable member.

FIG. 8A illustrates a side view of an embodiment of a system for modulating flow in a vessel.

FIG. 8B illustrates a perspective view of an embodiment of a system for modulating flow in a vessel.

FIG. 9 illustrates a cross-sectional plan view of at least a portion of an embodiment of a flow modulating device.

FIG. 10 illustrates a cross-sectional plan view of at least a portion of an embodiment of a flow modulating device.

FIG. 11 illustrates a cross-sectional plan view of at least a portion of an embodiment of a flow modulating device.

FIG. 12A illustrates a cross-sectional plan view of at least a portion of an embodiment of a flow modulating device.

FIG. 12B illustrates a cross-sectional plan view of at least a portion of an embodiment of a flow modulating device.

FIGS. 12C-12F illustrate cross-sectional views of at least a portion of various embodiments of flow modulating devices each having layers of material to couple the expandable member to a frame of the flow modulating device.

FIG. 13 is a schematic of an embodiment of a method of modulating flow in a vessel using any of the flow modulating devices described herein.

FIG. 14 is a schematic representation of portions of a human subject.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the contemplated embodiments. Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

In general, the systems and methods described herein may enable modulating and/or balancing of blood flow through a blood vessel. The modulating and/or balancing of blood flow may be performed by the devices described herein to occlude, partially occlude, and/or otherwise manage, modulate, or regulate blood flow to or through a portion of a blood vessel. In some examples, such modulation and/or balancing of blood flow to or through a blood vessel may result in additionally modulating pressure in the right atrium of the heart and/or other organs of the body.

The examples presented herein may relate to providing devices, methods, and/or methods of treatment (MOTs) for modulating and/or otherwise managing blood flow to or through one or more blood vessels. The terminology of restricting blood flow, regulating blood flow, modulating blood flow, managing blood flow, and balancing blood flow may be used to indicate regulating blood pressure, modulating blood pressure, managing blood pressure, and/or balancing blood pressure. As such, for example, a flow modulation device is synonymous with a pressure regulating device (i.e., a flow regulator is synonymous with a pressure regulator).

Managing blood flow through a blood vessel can be achieved by the devices described herein to provide an advantage of providing a plurality of flow modulation states. For example, the flow modulating devices described herein can include one or more expandable members that can each be inflated to one of a plurality of inflation states to modulate flow through the flow modulating device and therefore in a vessel within which the device is installed or in a vessel in fluid communication with the vessel in which the device is installed. The amount of flow and/or pressure in a vessel can be highly tuned or modulated based on the inflation state of each expandable member of the one or more expandable members. An inflation state of an expandable member may be based on a blood pressure in a vessel or another parameter of the vessel. A predetermined inflation state of an expandable member may be based on a blood pressure in the vessel, such that the pressure in the expandable member in the predetermined inflation state exceeds the blood pressure in the vessel. Additionally, or alternatively, a predetermined inflation state of an expandable member may be based on an inflation volume of the expandable member and/or a desired cross-sectional area reduction (or a desired cross-sectional area increase) of a cross-section of the lumen of a frame to which the expandable member is coupled.

Furthermore, the devices, systems, methods, and/or MOTs described herein can be used to solve a further technical problem of modulating flow in a vessel based on different blood pressures in the vessel or in an adjacent vessel. For example, any of the flow modulating devices and systems described herein can differentially modulate flow, for example based on whether the blood pressure is in a first blood pressure range, a second blood pressure range, a normal activity blood pressure range, an exertion-related blood pressure range, or any range therebetween. For example, flow modulating devices described herein can be configured to restrict blood flow through a blood vessel at elevated blood pressures or baseline activity blood pressures (e.g., from about 10 mmHg to about 25 mmHg, or from about 15 mmHg to about 25 mmHg), and also be configured to advantageously permit a larger amount of blood flow (by not restricting or minimally restricting blood flow through the blood vessel) at exertion-related blood pressures (e.g., about 25 mmHg to about 35 mmHg, greater than 25 mmHg, greater than 30 mmHg, or up to about 400 mmHg). In some cases, the flow modulating devices described herein can be configured to permit a larger amount of blood flow through the blood vessel at exertion-related blood pressures (e.g., greater than 25 mmHg, or greater than 30 mmHg), which can advantageously provide a patient with sufficient blood flow through the blood vessel during periods of exercise or stress to prevent negative side effects (e.g., fainting).

In some examples, the devices, methods, and/or MOTs described herein may be utilized to solve a technical problem of unwanted pressure increases in the right atrium in patients that have chronic kidney disease (CKD) and/or heart failure (HF). For example, patients with CKD and/or HF may exhibit reduced kidney function when pressure in the right atrium of the heart is above a predefined pressure threshold. The predefined pressure threshold may be used as a basis to determine whether a patient is exhibiting low vessel pressure (e.g., below a predefined pressure threshold) or high vessel pressure (e.g., above a predefined pressure threshold). When vessel pressure is determined to be high, the devices, methods, and/or MOTs can provide a technical solution to the technical problem recited above. For example, each of the devices described herein may be used to decrease pressure within one or more vessels to avoid right atrium pressure increases and/or pressure variations. In particular, the devices, methods, and/or MOTs described herein can be used to reduce and maintain low pressure in the right atrium, which provides a technical effect of enabling the kidneys to more effectively filter blood.

In addition, the devices, methods, and/or MOTs described herein can solve a further technical problem of accumulation of blood in the venous system. For example, the devices described herein may be used to reduce the accumulation of blood in the venous system, which can provide an advantage and technical effect of ensuring that pressure is not increased in the IVC. Such devices can advantageously eliminate excessive hospital readmissions and/or can provide for a long-term blood flow management therapy, improving both quality of life and overall survival rates and with a lower cost to a healthcare system.

Furthermore, the devices, methods, and/or MOTs described herein can be used to solve a further technical problem of regulating blood flow return, thus further mitigating pressure build-up in the right atrium. The examples described herein can perform blood flow management actively and/or passively to assist in reducing and/or maintaining right atrium pressures to a relatively low pressure even when a surge in blood volume occurs in one or more vessels of the venous system.

In some examples, the devices, methods, and/or MOTs described herein can be used to solve a further technical problem of exertion-related blood pressure in patients that have a flow restrictor implanted within a blood vessel. For example, flow restrictor devices described herein can be configured to restrict blood flow through a blood vessel at baseline or elevated blood pressures or normal activity blood pressures (e.g., from about 10 mmHg to about 25 mmHg, or from about 15 mmHg to about 25 mmHg), and also be configured to advantageously permit a larger amount of blood flow (by not restricting or minimally restricting blood flow through the blood vessel) at exertion-related blood pressures (e.g., greater than 25 mmHg, or greater than 30 mmHg). In some cases, the flow restrictors described herein can be configured to permit a larger amount of blood flow through the blood vessel at exertion-related blood pressures (e.g., greater than 25 mmHg, or greater than 30 mmHg), which can advantageously provide a patient with sufficient blood flow through the blood vessel during periods of exercise or stress to prevent negative side effects (e.g., fainting).

In some cases, patients who suffer from congestive heart failure (CHF) can also experience impaired renal function. Impaired renal function can be caused by increased systemic venous congestion as a result of low cardiac output and low blood pressure. The renal pressure gradient (between the renal arteries and renal veins) may be decreased due to elevated renal venous pressure, lowering glomerular filtration rate (GFR). GFR is the rate at which the kidney filters blood, for example, below 90 mL/min, which can be indicative of chronic kidney disease (CKD) that may eventually lead to end stage renal failure. Thus, reduction of renal venous pressure may improve GFR and reduce blood volume retention. Nevertheless, it may be desirable for a device (e.g., a flow modulating device described herein) configured to limit central venous volume to operate in a bi-modal fashion, configured to reduce venous pressure when the patient is at rest (e.g., under normal, baseline, or elevated blood pressure ranges), yet allow undisturbed or minimally disturbed venous flow when the patient exercises (or experiences an exertion-related blood pressure), so as to meet the dynamic flow/pressure requirements. Moreover, any such solution, when provided as an implantable device, can be percutaneously deliverable and can operate in a manner that minimizes risk of thrombosis.

Disclosed herein are systems and methods for a flow modulating device for a blood vessel. In some examples, the implantable flow modulating devices (or flow restrictors) described herein may be used in blood flow occlusion therapy. For example, the devices described herein may relate to venous occlusion therapy using implantable and/or electronically controlled flow modulating devices for the treatment of acute heart failure. Some devices may be non-implantable or partially implantable. Some devices operate without any powered input and are triggered by changes in anatomy and/or changes in physiology.

### Systems and Devices

The systems and devices described herein function to modulate blood flow in a vessel. In some embodiments, the systems and devices described herein can function to reduce systemic venous congestion, reduce renal venous pressure, improve glomerular filtration rate (GFR), and/or reduce blood volume retention. The system and devices are used for intravascular therapy, but can additionally, or alternatively, be used for any suitable applications, clinical or otherwise. The systems and devices can be configured and/or adapted to function for any suitable flow modulation function in a vessel.

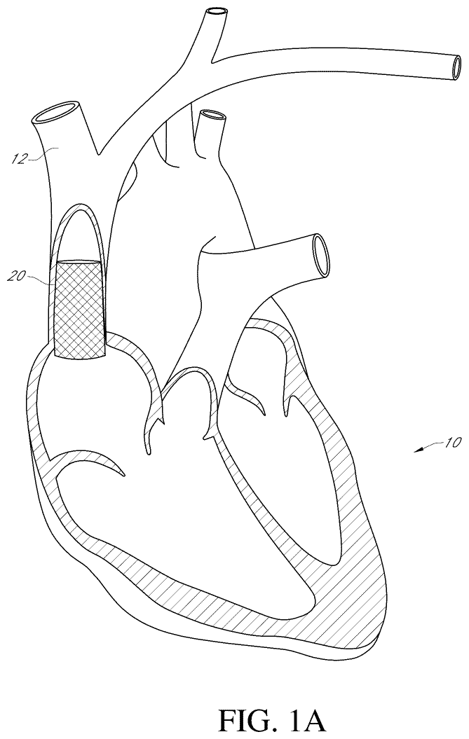

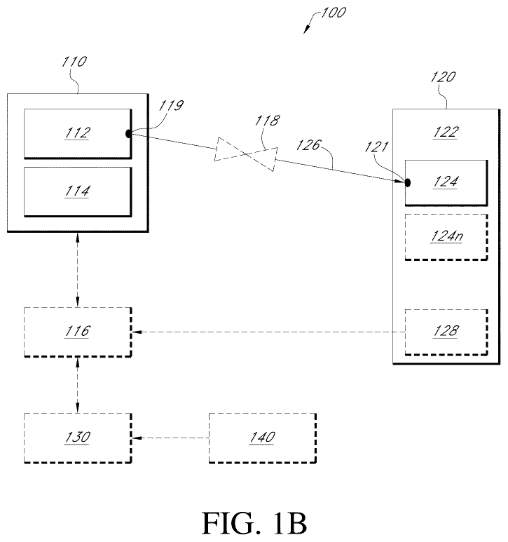

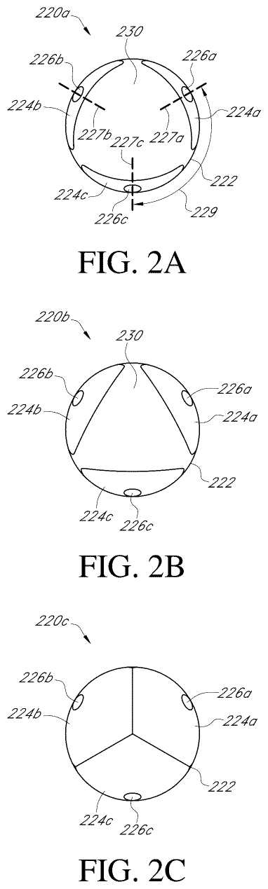

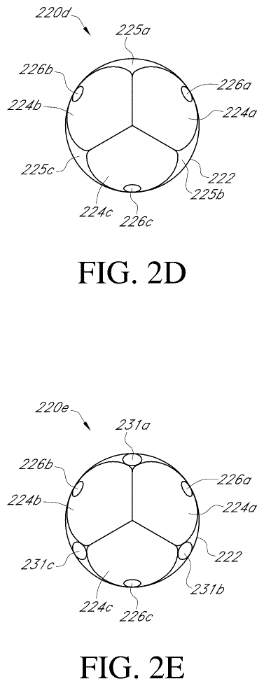

FIG. 1A illustrates an embodiment of a flow modulating device positioned in a vessel. An exemplary, non-limiting flow modulating device 20 is shown positioned in a Superior Vena Cava 12 (SVC) 12 of a heart 10. Although the flow modulating device 20 is shown as positioned in an SVC, one of skill in the art will appreciate that a flow modulating device, such as any of the flow modulating devices described herein or portions thereof (e.g., shown in FIGS. 1B-12F) may be positioned in any bodily lumen or vessel (e.g., inferior vena cave, SVC, renal artery, renal vein, etc.) to regulate flow therethrough or through an adjacent vessel fluidly connected to the vessel in which the device is positioned. A flow modulating device 20 may include a frame having an inflow end and an outflow end. The frame defines a lumen including an internal surface between the inflow end and the outflow end. One or more expandable members are coupled to at least one portion of the internal surface of the frame, as shown in FIGS. 2A-11. Each expandable member includes an inflation port for receiving an inflation fluid therethrough to inflate the expandable member. The one or more expandable members each define a volume configured to receive the inflation fluid therein through the inflation port to inflate the expandable member. The one or more expandable members are each reversibly inflatable to a plurality of inflation states to partially or fully occlude the lumen of the frame. For example, a volume defined by an expandable member, in an unrestricted flow state, may be empty or have substantially no inflation fluid in the volume defined by the expandable member. Further, for example, a volume defined by an expandable member, in a restricted flow state, may include inflation fluid in the volume and/or be substantially full of an inflation fluid in the volume defined by the expandable member. Still further, for example, in an intermediate or partial flow restricted state, a volume defined by the expandable member may be partially full (or partially empty) such that an inflation fluid is used to partially fill the volume defined by the expandable member. Although a restricted flow state, an unrestricted flow state, and an intermediate flow state are described, one of skill in the art will appreciate that any number of intervening flow states between the aforementioned flow states is also possible and contemplated herein.

In some variations, the one or more expandable members can include an expandable balloon. In some embodiments, the one or more expandable members can include or comprise a compliant material. In some embodiments, the one or more expandable members can be formed of a compliant material. In some embodiments, the one or more expandable members can consist essentially of a compliant material. A compliant material may exhibit a burst pressure of about 0 atmospheres (atm) to about 2 atm. In some embodiments, a compliant material may be able to expand about 20% to about 500%. Non-limiting examples of compliant materials include silicones, latex, polyvinyl chloride, polyolefin copolymer, or a combination thereof.

In some instances, the one or more expandable members can include or comprise a semi-compliant material. In some embodiments, the one or more expandable members can be formed of a semi-compliant material. In some embodiments, the one or more expandable members can consist essentially of a semi-compliant material. A semi-compliant material may exhibit a burst pressure of about 1 atm to about 25.5 atm. In some embodiments, a semi-compliant material may be able to expand about 10% to about 20%. Non-limiting examples of semi-compliant materials include polyethylene terephthalate, nylons, thermoplastic polyurethanes, thermoplastic elastomers, or a combination thereof.

In some instances, the one or more expandable members can include or comprise a non-compliant material. In some embodiments, the one or more expandable members can be formed of a non-compliant material. In some embodiments, the one or more expandable members can consist essentially of a non-compliant material. A non-compliant material may exhibit a burst pressure of about 1 atm to about 25.5 atm. In some embodiments, a non-compliant material may be able to expand about 0% to about 10%. Non-limiting examples of non-compliant materials include polyethylene terephthalate and like materials.

In some embodiments, one or more of the expandable members comprise a compliant material, and one or more of the expandable members comprise a non-compliant material. Expandable members having different properties or including different materials may, for example, achieve different filling rates of the expandable members, make a subset of the one or more expandable members more resistant to bursting, achieve various degrees of expansion of the one or more expandable members, allow differential filling of a cross-sectional area of a lumen of the frame based on a material of the one or more expandable materials, and the like. In some embodiments, one or more of the expandable members may include a blend of a compliant material and a semi-compliant material. In some embodiments, one or more of the expandable members may include a blend of a non-compliant material and a semi-compliant material. In some embodiments, one or more of the expandable members may include a blend of a compliant material and a non-compliant material.

In some embodiments, at least one expandable member of the one or more expandable members is formed of or comprises two or more expandable layers. For example, the two or more expandable layers may be coupled together such that a pocket forms between at least two of the two or more expandable layers. The pocket defines the volume that receives the inflation fluid therein. The two or more expandable layers may be formed of or comprise a compliant, semi-compliant, or non-compliant material, as described above. In some variations, one or more layers may include a compliant material and one or more layers can include a non-compliant material, such that materials are intermixed in any one of the one or more expandable members. In some variations, one or more layers may include a compliant material and one or more layers can include a semi-compliant material, such that materials are intermixed in any one of the one or more expandable members. In some variations, one or more layers may include a semi-compliant material and one or more layers can include a non-compliant material, such that materials are intermixed in any one of the one or more expandable members.

In some variations, the frame is an intraluminal device, a stent, a braided tubular or ovular structure, or the like. The frame can include one or more coatings or coverings thereon. For example, at least a portion of the frame may be covered in or coated in a polymer, a biomaterial, a textile, a drug, or the like. Further, for example, at least a portion of the frame can include one or more layers of material to facilitate coupling the one or more expandable members to the frame.

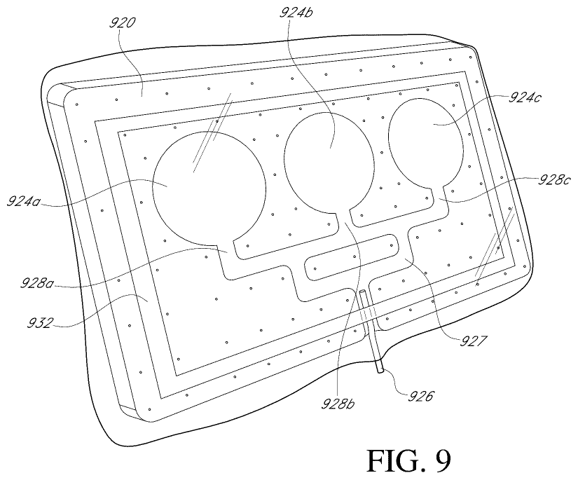

As will be described in greater detail below with respect to FIGS. 9-12F, the one or more layers of material on at least a portion of the frame may be used to couple the one or more expandable members to the frame. In some variations, coupling the one or more expandable members directly to at least a portion of the frame causes the one or more expandable members to be irreversibly stretched, damaged, or punctured. A technical solution for this technical problem is to include a layer of material in the coupling of the frame to the one or more expandable members. For example, one or more layers of material may be directly attached to at least a portion of the frame and the one or more expandable members may be coupled to at least a portion of the one or more layers of material (and thus indirectly attached to at least a portion of the frame). Further, the one or more expandable members may be directly attached to at least a portion of the frame and stabilized by one or more layers coupled to the one or more expandable members (and thus indirectly attached to at least a portion of the frame). Still further, the one or more expandable members may be sandwiched between two or more layers of material that are coupled to at least a portion of the frame, to secure the one or more expandable members to at least a portion of the frame. The one or more layers of material can include or comprise a polymer, a biomaterial, a woven material, or a textile. The one or more layers of material can be formed of a polymer, a biomaterial, a woven material, or a textile. The one or more layers of material can consist essentially of a polymer, a biomaterial, a woven material, or a textile.

In some variations, a layer of material is embedded between the two or more expandable layers to couple the one or more expandable members (formed by the two or more expandable layers) to at least a portion of the frame, as will be described in greater detail in connection with FIGS. 9-12F.

In some embodiments, the frame, the one or more expandable members, and/or the optional one or more layers of material can include an embedded radiopaque marker. The use of the embedded radiopaque marker may increase visibility of the device (e.g., device 20) using fluoroscopy during device placement in a vessel, repositioning the device in a vessel, extracting a device from a vessel, and/or routine maintenance or check-ups on the device and/or the patient.

FIG. 1B shows a schematic of an embodiment of a system 100 for modulating flow in a vessel. The system 100 includes an inflation apparatus 110 and a flow modulating device 120. The system 100 functions to deliver inflation fluid from an inflation apparatus 110 to a flow modulating device 120, via inflation line 126, to cause one or more expandable members 124 coupled to at least a portion of a frame 122 of a flow modulating device 120 to expand to partially or fully occlude a lumen of the frame 122 and thus a vessel in which the flow modulating device 120 is positioned.

The inflation apparatus 110 includes a reservoir 112 and a pump 114. In general, the reservoirs described herein may be configured to contain inflation fluid. For example, the reservoirs may include walls, ends, or portions that are sealed or at least partially sealed to receive and hold a volume of inflation fluid. The pump 114 can be a motorized pump (e.g., a positive-displacement pump, an impulse pump, a velocity pump, a steam pump, a valveless pump, and/or other pump using electrical or inductive power) or a manual pump, for example like a syringe. The pump 114 acts upon reservoir 112 to cause inflation fluid to flow into a first end 119 of inflation line 126. An optional valve 118 (manual or motorized valve) can be actuated to allow inflation fluid to flow through inflation line 126 or to inhibit inflation fluid to flow through inflation line 126. The optional valve 118 may be a one-way valve (e.g., a check valve) or a multi-way valve (e.g., including two or more ports). A second end 121 of inflation line 126 is fluidly connected to an inflation port of at least one expandable member 125 or one or more inflation ports of one or more expandable members 124n (n equaling any number between one and twenty, one and fifteen, one and twelve, one and ten, etc.) that are coupled to at least a portion of a frame 122 of flow modulating device 120. The inflation fluid flows into a volume defined by an expandable member 124. The inflation fluid fills at least a portion of the volume defined by the expandable member 124 to cause the expandable member 124 to reversibly inflate and at least partially occlude a lumen of a frame 122 of the flow modulating device 120, which may at least partially occlude a vessel in which the flow modulating device 120 is positioned. The inflation fluid can include a gas, saline, heparinized saline, blood, contrast, and the like, or a combination thereof.

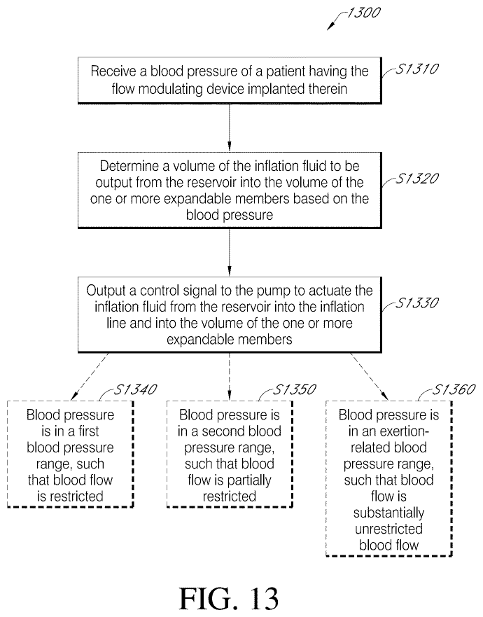

Flow modulating device 120 may optionally include an optional sensor 128 for sensing one or more blood flow or pressure properties in the vessel. For example, the sensor 128 can include one or more of a strain gauge, a piezoelectric sensor, a capacitance sensor, and a vacuum pressure sensor, such that the sensor 128 senses a pressure in the blood vessel. The optional sensor 128 may be communicatively coupled (e.g., wired connection or wireless connection) to an optional processor 116. The processor 116 may execute one or more methods or sets of instructions as will be described in greater detail in connection with FIG. 13 and the associated written description. The optional processor 116 may be communicatively coupled to an optional antenna 130 for receiving instructions and/or sensor data (e.g., from an optional computing device 140, inflation apparatus 110, optional sensor 128, etc.) and transmitting instructions and/or sensor data (e.g., to an optional computing device 140, inflation apparatus 110, flow modulating device 120, etc.). Optional computing device 140 can be a local computing device, for example worn on, attached to, embedded in, or otherwise coupled to a user. Optional computing device 140 can be a remote computing device, for example a server, workstation, physician computing device, etc.

In some embodiments, the inflation apparatus 110 is positioned subcutaneously in a patient; and the flow modulating device 120 is implanted intraluminally. Alternatively, the inflation apparatus 110 may be externally coupled to a patient. In further embodiments, the inflation apparatus 110 may be implanted in a patient.

In at least some instances, the one or more expandable members 124 may not restrict flow or may not sufficiently restrict flow through a frame 122 of the flow modulating device 120 when a fluid pressure in the one or more expandable members 124 is less than or equal to a blood pressure of the patient in which the flow modulating device 120 is positioned. A technical solution for this technical problem is to modulate a fluid pressure in the one or more expandable members 124 based on a blood pressure of the patient. For example, the inflation apparatus 110 can deliver the inflation fluid to the volume defined by the one or more expandable members 124 such that a fluid pressure in the volume is substantially equal to or greater than a blood pressure of the patient in which the device is positioned. For example, the inflation apparatus 110 can deliver the inflation fluid to the volume defined by the one or more expandable members 124 such that a fluid pressure in the volume is at least about 10 mmHg. Further, for example, the inflation apparatus 110 can deliver the inflation fluid to the volume defined by the one or more expandable members 124 such that a fluid pressure in the volume is between about 10 mmHg and about 40 mmHg.

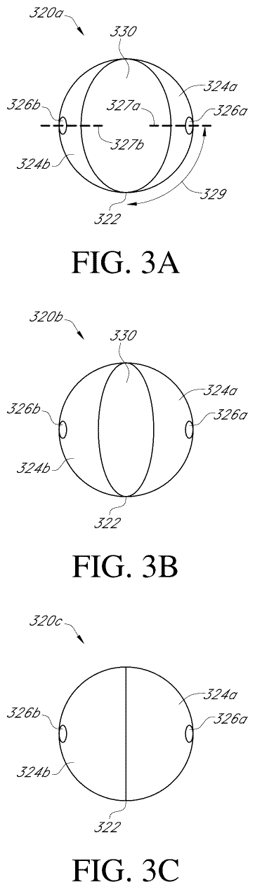

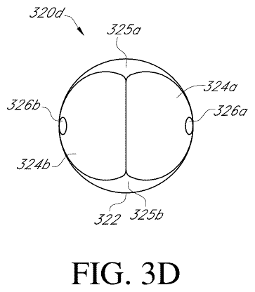

FIGS. 2A-2C illustrate cross-sectional views of an embodiment of a flow modulating device in a plurality of inflation states. For example, flow modulating device 220a is shown in a first inflation state (FIG. 2A), in which the lumen 230 of the frame 222 is substantially open, un-occluded, unrestricted, etc. to enable substantially uninhibited blood flow therethrough. As shown in FIG. 2A, the expandable members 224a, 224b, 224c of flow modulating device 220a are uninflated or substantially uninflated such that the expandable members 224a, 224b, 224c do not substantially impinge on the lumen 230 of frame 222. Flow modulating device 220b is shown in a second inflation state (FIG. 2B), in which the lumen 230 of the frame 222 is partially open, partially occluded, partially restricted, etc. (cross-sectional area of the lumen is reduced) to partially modulate or disturb blood flow therethrough. As shown in FIG. 2B, the expandable members 224a, 224b, 224c of flow modulating device 220b are partially inflated or partially filled with inflation fluid such that the expandable members 224a, 224b, 224c partially impinge on or partially occlude or fill the lumen 230 of frame 222. Flow modulating device 220c is shown in a third inflation state (FIG. 2C), in which the lumen 230 (not labeled in FIG. 2C) of the frame 222 is substantially or fully closed, occluded, restricted, etc. (cross-sectional area of the lumen is further reduced as compared to FIG. 3B) to substantially or fully restrict blood flow therethrough. As shown in FIG. 2C, the expandable members 224a, 224b, 224c of flow modulating device 220c are fully inflated or substantially inflated or substantially filled with inflation fluid such that the expandable members 224a, 224b, 224c impinge on or fully occlude or substantially occlude the lumen 230 of frame 222. In the example of FIG. 2C, one or more of the expandable members 224a, 224b, 224c may contact one or more of the other expandable members 224a, 224b, 224c to occlude the lumen 230.

Although the inflation states are described as first, second, and third, one of skill in the art will appreciate that these are used simply for ease of description and do not suggest an order or sequence of the inflation states. Further, although three inflation states are shown, one of skill in the art will appreciate that any number of inflation states are possible. For example, when a flow modulating device includes more than one expandable member, each expandable member can be inflated to an inflation state that is independent from and/or different from the other expandable members of the one or more expandable members. Further for example, one expandable member (or more than one expandable member) can each receive a predefined or predetermined volume of inflation fluid such that the volume is proportional to the inflation state when there is one expandable member. Further, when more than one expandable member is included in a device, each expandable member can receive an independent and/or different volume of inflation fluid than the other expandable members.

Flow modulating devices 220a, 220b, 220c each include a frame 222 and a lumen 230 defined by the frame 222 (although lumen 230 of flow modulating device 220c is not labeled since the lumen 230 is substantially occluded). Each expandable member 224a, 224b, 224c is coupled to at least a portion of the frame 222. Alternatively, at least one expandable member is coupled to at least a portion of the frame 222 and the other expandable members are coupled to at least a portion of the at least one expandable member. Flow modulating devices 220a, 220b, 220c each include expandable members 224a, 224b, 224c. Expandable member 224a includes inflation port 226a for receiving an inflation fluid therethrough to inflate the expandable member. Similarly, expandable member 224b includes inflation port 226b and expandable member 224c includes inflation port 226c.

In some embodiments, as shown in FIG. 2A, a first expandable member 224a is circumferentially offset 229 from a second expandable member 224b and/or a third expandable member 224c along the internal surface of the frame 222. The circumferential offset 229 may be measured from a central axis of each expandable member. For example, expandable member 224a has central axis 227a; expandable member 224b has central axis 227b; and expandable member 224c has central axis 227c. Each central axis 227a, 227b, 227c may be positioned similarly to a radius of a lumen 230 of the frame 222. The circumferential offset 229 can be between about 60 degrees and about 150 degrees; about 100 degrees to about 140 degrees; about 30 degrees to about 200 degrees; about 150 degrees to about 200 degrees; about 50 degrees to about 260 degrees; etc. In some embodiments, the circumferential offset may be substantially equal to 360 degrees divided by a number of expandable members at least partially disposed in the frame. In some embodiments, the circumferential offset may be any number of degrees that achieves the intended partial or full occlusion of the lumen of the frame. Expandable members 224a, 224b, 224c may be concomitantly filled with inflation fluid through their respective inflation ports. Expandable members 224a, 224b, 224c may be sequentially filled with inflation fluid through their respective inflation ports. Expandable members 224a, 224b, 224c may be filled with inflation fluid in a predefined or predetermined sequence through their respective inflation ports.

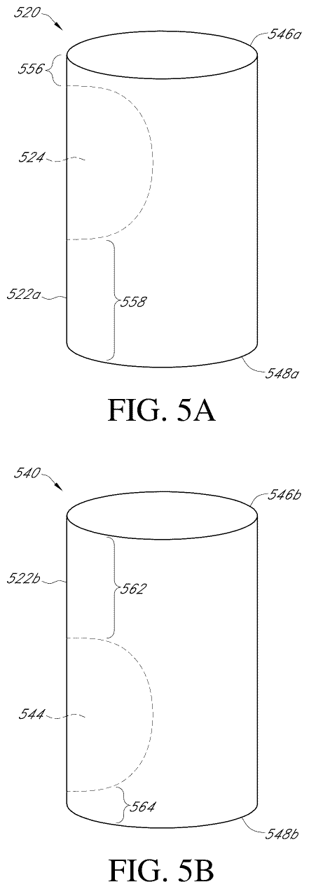

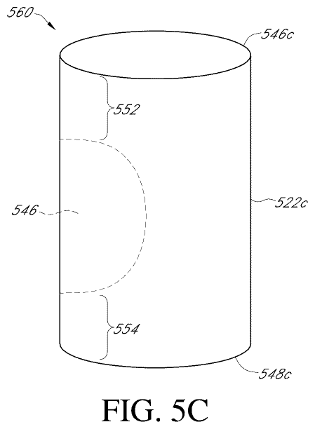

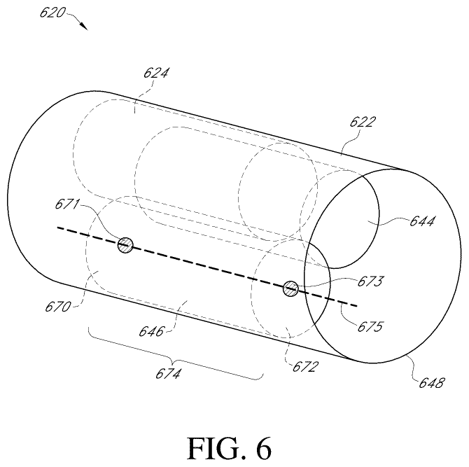

In some embodiments, when a flow modulating device is in a partially or substantially fully closed, occluded, or restricted state (e.g., one or more expandable members are partially or fully inflated), blood may pool, exhibit stasis, or create eddies at or proximal to an upstream or inflow end of the flow modulating device. For example, these stasis zones may be where the expandable member couples to the frame and the like. For example, these stasis zones may be where the expandable member and frame together define a pocket, groove, indentation, or concave section and the like. For example, these stasis zones may be where a first expandable member contacts a second expandable member and the like. To alleviate blood stasis within a potential stasis zone, a flow modulating device may include one or more stasis reduction solutions. For example, a flow modulating device 200d, for example as shown in FIG. 2D, may include one or more stasis reduction flow paths 225a, 225b, 225c positioned within or adjacent to one or more potential stasis zones to ensure that blood may flow through the one or more of the potential stasis zones rather than pool or exhibit stasis. In particular, an outer surface (i.e., external surface of the expandable member) of an expandable member 224a (or 224b or 224c) and at least a portion of an inner surface of the frame 222 together define the stasis reduction flow path 225. The stasis reduction flow path 225 may extend along at least a portion of a longitudinal length (e.g., substantially parallel to axis 675 of FIG. 6) of the frame 222, fluidly connecting the inflow end to the outflow end (shown in FIGS. 5A-5C). Alternatively, as shown in flow modulating device 200e of FIG. 2E, one or more longitudinally extending conduits 231a, 231b, 231c (e.g., substantially parallel to axis 675 of FIG. 6) may be coupled to at least a portion of an inner surface of the frame 222 between the one or more expandable members 224a (or 224b or 224c) to provide a stasis reduction flow path. The conduits 231 defining the stasis reduction flow path may extend along at least a portion of a longitudinal length (e.g., substantially parallel to axis 675 of FIG. 6) of the frame 222, fluidly connecting the inflow end to the outflow end (shown in FIGS. 5A-5C).

Although a number of stasis reduction flow paths is shown equaling a number of expandable members, one of skill in the art will appreciate that any number of stasis reduction flow paths are possible, less than, greater than, or equal to the number of expandable members. For example, a flow modulating device may include one, more than one, one or more, or a plurality (e.g., one, one to five, one to 10, five to 10, etc.) of stasis reduction flow paths.

FIGS. 3A-3C illustrate cross-sectional views of an embodiment of a flow modulating device in a plurality of inflation states. To illustrate that any number of expandable members may be used, FIGS. 3A-3C show a flow modulating device with two expandable members, as compared to the three expandable members in FIGS. 2A-2C. For example, flow modulating device 320a is shown in a first inflation state (FIG. 3A), in which the lumen 330 of the frame 322 is substantially open, un-occluded, unrestricted, etc. to enable substantially uninhibited blood flow therethrough. As shown in FIG. 3A, the expandable members 324a, 324b of flow modulating device 320a are uninflated or substantially uninflated such that the expandable members 324a, 324b do not substantially impinge on the lumen 330 of frame 322. Flow modulating device 320b is shown in a second inflation state (FIG. 3B), in which the lumen 330 of the frame 322 is partially open, partially occluded, partially restricted, etc. (cross-sectional area of the lumen is reduced) to partially modulate or disturb blood flow therethrough.

As shown in FIG. 3B, the expandable members 324a, 324b of flow modulating device 320b are partially inflated or partially filled with inflation fluid such that the expandable members 324a, 324b partially impinge on or partially occlude or fill the lumen 330 of frame 322. Flow modulating device 320c is shown in a third inflation state (FIG. 3C), in which the lumen 330 (not labeled in FIG. 3C) of the frame 322 is substantially or fully closed, occluded, restricted, etc. (cross-sectional area of the lumen is further reduced as compared to FIG. 3B) to substantially or fully restrict blood flow therethrough. As shown in FIG. 3C, the expandable members 324a, 324b of flow modulating device 320c are fully inflated or substantially inflated or substantially filled with inflation fluid such that the expandable members 324a, 324b impinge on or fully occlude or substantially occlude the lumen 330 of frame 322. In the example of FIG. 3C, an expandable member 324a, 324b may contact the other expandable member 324a, 324b to occlude the lumen 330.

Although the inflation states are described as first, second, and third, one of skill in the art will appreciate that these are used simply for ease of description and do not suggest an order or sequence of the inflation states. Further, although three inflation states are shown, one of skill in the art will appreciate that any number of inflation states are possible, as described above.

Flow modulating devices 320a, 320b, 320c each include a frame 322 and a lumen 330 defined by the frame 322 (although lumen 330 of flow modulating device 320c is not labeled since the lumen 330 is substantially occluded). Flow modulating devices 320a, 320b, 320c each include expandable members 324a, 224b. Each expandable member 324a, 324b is coupled to at least a portion of the frame 322. Alternatively, at least one expandable member is coupled to at least a portion of the frame 322 and the other expandable member is coupled to at least a portion of the at least one expandable member. Expandable member 324a includes inflation port 326a for receiving an inflation fluid therethrough to inflate the expandable member. Similarly, expandable member 324b includes inflation port 326b.

In some embodiments, as shown in FIG. 3A, a first expandable member 324a is circumferentially offset 329 from a second expandable member 324b along the internal surface of the frame 322. The circumferential offset 329 may be measured from a central axis of each expandable member. For example, expandable member 324a has central axis 327a; and expandable member 324b has central axis 327b. Each central axis 327a, 327b may be positioned similarly to a radius of a lumen 330 of the frame 322. The circumferential offset 329 can be between about 100 degrees and about 220 degrees; about 150 degrees and about 200 degrees; etc. Expandable members 324a, 324b may be concomitantly filled with inflation fluid through their respective inflation ports. Expandable members 324a, 324b may be sequentially filled with inflation fluid through their respective inflation ports. Expandable members 324a, 324b may be filled with inflation fluid in a predefined or predetermined sequence through their respective inflation ports. The one or more expandable members of FIGS. 2A-3C may also be longitudinally offset from the other expandable member and/or an end of the frame, as described in greater detail in FIGS. 5A-5C.

FIG. 3D shows another example of a flow modulating device 320d including one or more stasis reduction flow paths 325a, 325b. An outer surface of an expandable member 324a and/or 324b and at least a portion of an inner surface of the frame 322 together define the stasis reduction flow path 325. The stasis reduction flow path 325 may extend along at least a portion of a longitudinal length (e.g., substantially parallel to axis 675 of FIG. 6) of the frame 322, fluidly connecting the inflow end to the outflow end (shown in FIGS. 5A-5C).

Although conduits functioning as stasis reduction flow paths are not shown with respect to the embodiment of FIGS. 3A-3D, one of skill in the art will appreciate that the conduits of FIG. 2E can be applied to the embodiment in FIG. 3D. Further, although a number of stasis reduction flow paths is shown equaling a number of expandable members, one of skill in the art will appreciate that any number of stasis reduction flow paths are possible, less than, greater than, or equal to the number of expandable members. For example, a flow modulating device may include one, more than one, one or more, or a plurality (e.g., 1, 1-5, 1-10, 5-10, etc.) of stasis reduction flow paths.

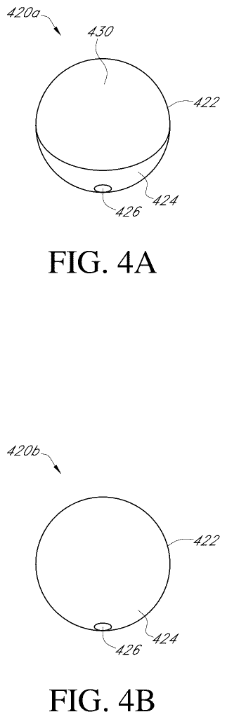

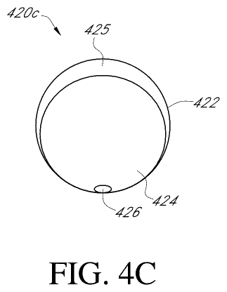

FIGS. 4A-4B illustrate cross-sectional views of an embodiment of a flow modulating device in a plurality of inflation states. To illustrate that any number of expandable members may be used, FIGS. 4A-4B show a flow modulating device with one expandable member, as compared to the three expandable members in FIGS. 2A-2C and two expandable members in FIGS. 3A-3C, although any number can be used (e.g., one, two, three, four, five, one to 10, greater than 10, etc.). For example, flow modulating device 420a is shown in a first inflation state (FIG. 4A), in which the lumen 430 of the frame 422 is substantially open, un-occluded, unrestricted, etc. to enable substantially uninhibited blood flow therethrough. As shown in FIG. 4A, the expandable member 424 of flow modulating device 420a is uninflated or substantially uninflated such that the expandable member 424 does not substantially impinge on the lumen 430 of frame 422. Flow modulating device 420b is shown in a second inflation state (FIG. 4B), in which the lumen 430 (not labeled in FIG. 4B) of the frame 422 is substantially or fully closed, occluded, restricted, etc. (cross-sectional area of the lumen is further reduced as compared to FIG. 4A) to substantially or fully restrict blood flow therethrough. As shown in FIG. 4B, the expandable member 424 of flow modulating device 420b is fully inflated or substantially inflated or substantially filled with inflation fluid such that the expandable member 424 impinges on or fully occludes or substantially occludes the lumen 430 of frame 422. In the example of FIG. 4B, an expandable member 424 may contact an opposite side of the lumen 430 of the frame 422 to occlude the lumen 430.

Although the inflation states are described as first and second, one of skill in the art will appreciate that these are used simply for ease of description and do not suggest an order or sequence of the inflation states. Further, although two inflation states are shown, one of skill in the art will appreciate that any number of inflation states are possible (e.g., one to three, one to 10, greater than 10, 10 to 10, etc.), as described above.

Further, as shown in FIG. 4A, the expandable member 424 can extend at least partially along a circumference of the internal surface of the frame, although a more focal coupling to a least a portion of the internal surface of the frame 422 is also contemplated. For example, expandable member 424 extends about 20 degrees to about 210 degrees; about 150 degrees to about 200 degrees; etc. of the circumference of at least a portion of the internal surface of the frame 422.

Flow modulating devices 420a, 420b each include a frame 422 and a lumen 430 defined by the frame 422 (although lumen 430 of flow modulating device 420b is not labeled since the lumen 430 is substantially occluded). Flow modulating devices 420a, 420b each include an expandable member 424. Expandable member 424 is coupled to at least a portion of the frame 422. Expandable member 424 includes inflation port 426 for receiving an inflation fluid therethrough to inflate the expandable member 424.

FIG. 4C shows another example of a flow modulating device 420c including a stasis reduction flow path 425. An outer surface of an expandable member 424 and at least a portion of an inner surface of the frame 422 together define the stasis reduction flow path 425. The stasis reduction flow path 425 may extend along at least a portion of a longitudinal length (e.g., substantially parallel to axis 675 of FIG. 6) of the frame 422, fluidly connecting the inflow end to the outflow end (shown in FIGS. 5A-5C).

Although conduits functioning as stasis reduction flow paths are not shown with respect to the embodiment of FIGS. 4A-4C, one of skill in the art will appreciate that the conduits of FIG. 2E can be applied to the embodiment of FIG. 4C. Further, although a number of stasis reduction flow paths is shown equaling a number of expandable members, one of skill in the art will appreciate that any number of stasis reduction flow paths are possible, less than, greater than, or equal to the number of expandable members. For example, a flow modulating device may include one, more than one, one or more, or a plurality (e.g., one, one to five, one to 10, five to 10, etc.) of stasis reduction flow paths.

FIGS. 5A-5C illustrate various embodiments of flow modulating devices 520, 540, 560 having one or more expandable members 524, 544, 546, respectively, coupled to various portions of an internal surface of a frame 522a, 522b, 522c, respectively. As shown in FIG. 5A, flow modulating device 520 includes an expandable member 524 that is coupled to at least a portion of the internal surface of the frame 522a adjacent to the inflow end 546a of the frame. The inflow end 456a is opposite the outflow end 548a. For example, a first length 556 between the expandable member 524 and the inflow end 546a may be less than a second length between the expandable member 524 and the outflow end 548a.

As shown in FIG. 5B, flow modulating device 540 includes expandable member 544 that is coupled to at least a portion of the internal surface of the frame 522b adjacent to the outflow end 548b of the frame 522b. The outflow end 548b is opposite the inflow end 546b. For example, a first length 562 between the expandable member 544 and the inflow end 546a may be greater than a second length between the expandable member 544 and the outflow end 548b.

As shown in FIG. 5C, flow modulating device 560 include an expandable member 546 that is coupled to at least a portion of the internal surface of the frame 522c substantially equidistant (length 552 being substantially similar to or equal to length 554) between the inflow end 546c and the outflow end 548c of the frame 522c.

FIG. 6 shows a perspective view of an embodiment of a flow modulating device 620 having one or more cylindrically shaped expandable members 624, 644, 646. For example, at least one expandable member may be a cylindrical shape such that the expandable member extends between the inflow end 647 and the outflow end 648 of the frame 622. Said another way, a surface 674 of expandable member 646 extends between a first end 670 of the expandable member 646 and a second end 672 of the expandable member 646. Said still another way, the first end 670 is substantially parallel to a second end 672, and the surface 674 extending between the first and second ends 670, 672 and joining the first and second ends 670, 672 is a curved surface. One or both of the first and second ends 670, 672 can be circular in shape, although other shapes are also contemplated herein. In some embodiments, a center 671, 673 of the first and second ends 670, 672, respectively, can be aligned along an axis 675.

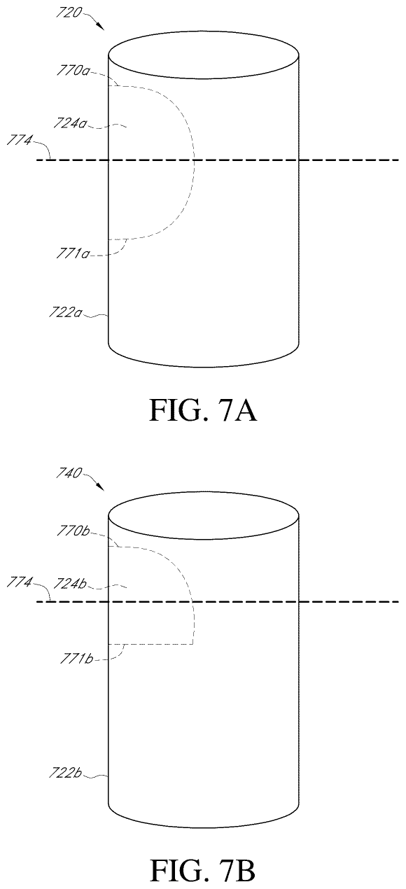

FIG. 7A shows a perspective view of an embodiment of an expandable member 724a of a flow modulating device 720. The expandable member 724a is coupled to at least a portion of frame 722a. The expandable member 724a includes a first end 770a opposite a second end 771a about a cross-sectional axis 774 of the expandable member 724a. The first end 770a is substantially convex, when fully or substantially inflated, and the second end 771a is substantially convex, when fully or substantially inflated. For example, the expandable member 724a is reminiscent of a balloon shape. In some embodiments, a shape of expandable member 724a and/or a volume defined by expandable member 724a in FIG. 7A may cause less blood flow disturbances (e.g., during partial occlusion and/or during minimal occlusion). In some embodiments, a shape of expandable member 724a and/or a volume defined by expandable member 724a in FIG. 7A may have minor blood stasis zones. In some embodiments, a shape of expandable member 724a and/or a volume defined by expandable member 724a in FIG. 7A may include more stability to support the elevation in blood pressure during at least partial occlusion.

FIG. 7B shows a perspective view of an embodiment of another shape of an expandable member 724b of a flow modulating device 740. The expandable member 724b is coupled to at least a portion of frame 722b. The expandable member 724b includes a first end 770b opposite a second end 771b about a cross-sectional axis 774 of the expandable member 724b. The first end 770b is substantially convex, when fully or substantially inflated, and the second end 771b is substantially planar, when fully or substantially inflated. For example, the expandable member 724b is reminiscent of a blister pack shape.

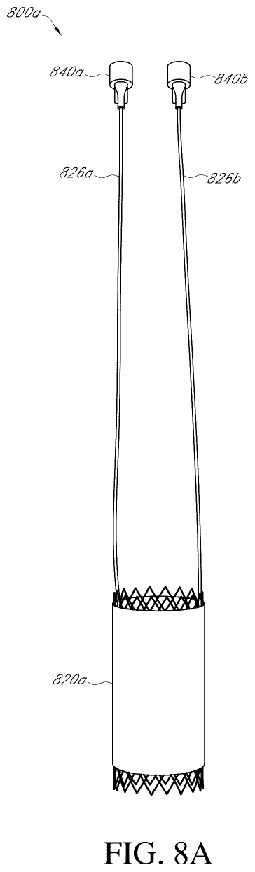

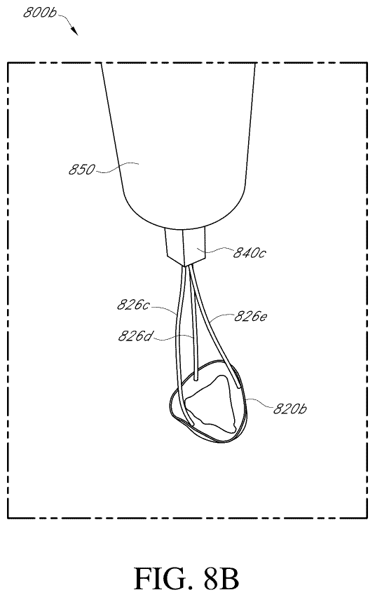

FIG. 8A shows a side view of an embodiment of a system 800a for modulating flow in a vessel. System 800a includes inflation lines 826a, 826b coupled to one or more expandable members positioned at least partially within a frame of flow modulating device 820a. For example, inflation line 826a may couple to a first expandable member, and inflation line 826b may couple to a second expandable member. Alternatively, inflation line 826a may couple to one or more expandable members, and inflation line 826b may couple to one or more expandable members. The one or more expandable members are inflated concomitantly, sequentially, or in a predefined or stochastic order. Connection 840a of inflation line 826a may fluidly connect inflation line 826a to a reservoir, and connection 840b of inflation line 826b may fluidly connect inflation line 826b to the same reservoir as inflation line 826a or a different reservoir. The reservoir may be connected to a manual or automatic pump, as described elsewhere herein. Connections 840a, 840b may be a twist to connect fitting, a snap-fit connection, and the like.

FIG. 8B shows a perspective view of an embodiment of a system 800b for modulating flow in a vessel. System 800b includes inflation lines 826c, 826d, 826e, each coupled to one or more expandable members in flow modulating device 820b. Inflation lines 826c, 826d, 826e are fluidly connected to reservoir 850, for example a syringe having a manual pump (i.e., plunger). As shown in FIG. 8B, inflation lines 826c, 826d, 826e may be fluidly coupled to reservoir 850 at common connection point 840c, although an individual connection point for each inflation line is also contemplated herein. Further, although FIG. 8A shows two inflation lines and FIG. 8B shows three inflation lines, one of skill in the art will appreciate that any number of inflation lines are contemplated herein. For example, there may be a 1:1 ratio of inflation lines to expandable members. Alternatively, there may be one inflation line to two or more or a plurality of expandable members. Still further for example, there may be two or more or a plurality of inflation lines for each expandable member.

FIGS. 9-11 show various configurations between inflation lines and expandable members in a flow modulating device. FIG. 9 shows a cross-sectional plan view of at least a portion 920 of an embodiment of a flow modulating device. For example, as shown in FIG. 9, inflation line 926 is directly fluidly connected (via common path 927) to the first inflation port 928a, the second inflation port 928b, and the third inflation port 928c of expandable members 924a, 924b, 924c, respectively. In the embodiment of FIG. 9, the first expandable member 924a, the second expandable member 924b, and the third expandable member 924c are configured to be inflated substantially concurrently or in parallel via inflation line 926. Although first, second, and third designations are used, one of skill in the art will appreciate that these designations are simply for ease of describing the embodiment and do not impart a sequence or order to the embodiment.

Further, as shown in FIG. 9 in at least some embodiments, the expandable members 924a, 924b, 924c are formed by two expandable layers (e.g., thermoplastic polyurethane). To couple the expandable layers to a frame, a layer of material 932 is positioned between the expandable layers, but absent from a volume defined by one or more expandable members. Sutures or other binding material may be passed through and/or applied to the layer of material 932 to secure the layer of material 932 and thus the expandable layers to a frame of a flow modulating device.

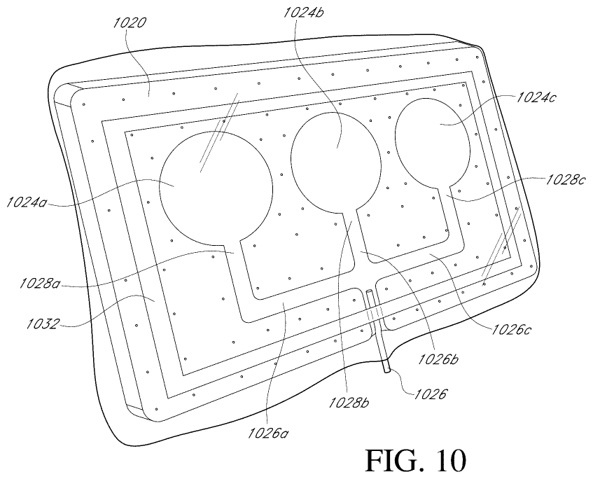

FIG. 10 shows a cross-sectional plan view of at least a portion 1020 of an embodiment of a flow modulating device. For example, as shown in FIG. 10, inflation line 1026 is directly fluidly connected to the first inflation port 1028a of expandable member 1024a, the second inflation port 1028b of expandable member 1024b, and the third inflation port 1028c of expandable member 1024c, via fluid paths 1026a, 1026b, 1026c, respectively. In the embodiment of FIG. 10, the first expandable member 1024a, the second expandable member 1024b, and the third expandable member 1024c are configured to be inflated substantially concurrently or in parallel via inflation line 1026 (and fluid paths 1026a, 1026b, 1026c). Although first, second, and third designations are used, one of skill in the art will appreciate that these designations are simply for ease of describing the embodiment and do not impart a sequence or order to the embodiment.

Further, as shown in FIG. 10 in at least some embodiments, the expandable members 1024a, 1024b, 1024c are formed by two expandable layers (e.g., thermoplastic polyurethane). To couple the expandable layers to a frame, a layer of material 1032 is positioned between the expandable layers, but absent from a volume defined by one or more expandable members. Sutures or other binding material may be passed through and/or applied to the layer of material 1032 to secure the layer of material 1032 and thus the expandable layers to a frame of a flow modulating device.

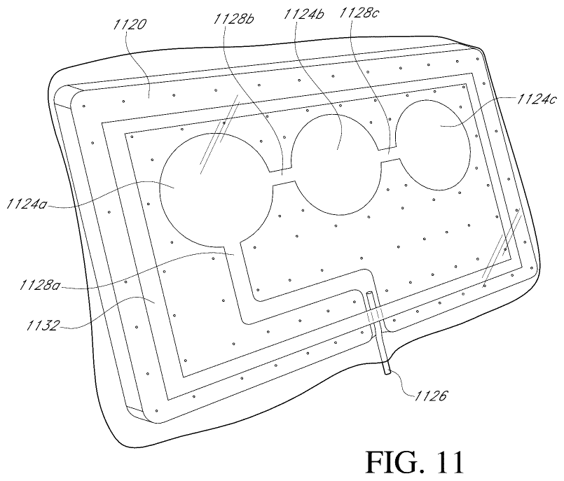

FIG. 11 shows a cross-sectional plan view of at least a portion 1120 of an embodiment of a flow modulating device. For example, as shown in FIG. 11, inflation line 1126 is directly fluidly connected to the first inflation port 1128a of expandable member 1024a; and indirectly fluidly connected to the second inflation port 1128b of expandable member 1124b and the third inflation port 1128c of expandable member 1124c. For example, inflation fluid flows from inflation line 1126 into the first expandable member 1124a through port 1128a, into the second expandable member 1124b through port 1128b, and into the third expandable member 1124c through port 1128c. In the embodiment of FIG. 11, the first expandable member 1124a, the second expandable member 1124b, and the third expandable member 1124c are configured to be inflated substantially sequentially or in series via inflation line 1126. Although first, second, and third designations are used, one of skill in the art will appreciate that these designations are simply for ease of describing the embodiment and do not impart a sequence or order to the embodiment.

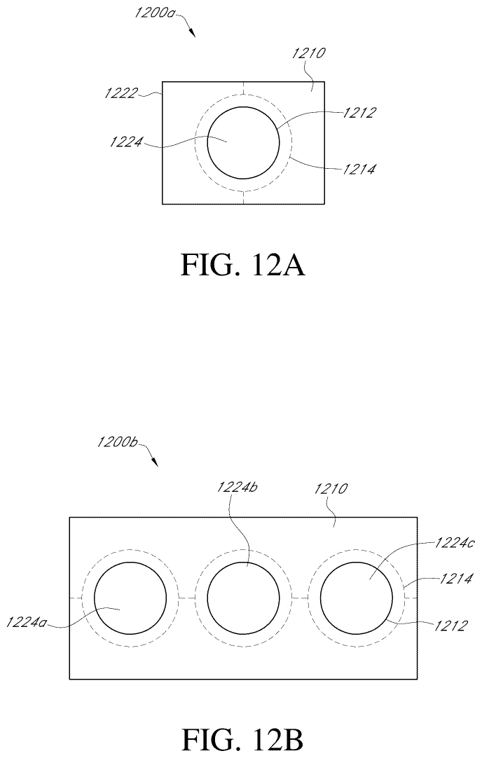

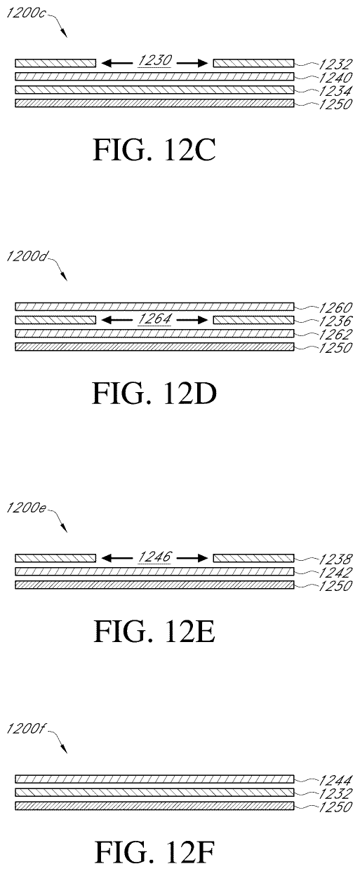

Further, as shown in FIG. 11 in at least some embodiments, the expandable members 1124a, 1124b, 1124c are formed by two expandable layers (e.g., thermoplastic polyurethane). To couple the expandable layers to a frame, a layer of material 1132 is positioned between the expandable layers, but absent from a volume defined by one or more expandable members. Sutures or other binding material may be passed through and/or applied to the layer of material 1132 to secure the layer of material 1132 and thus the expandable layers to a frame of a flow modulating device. Further, although three expandable members are shown in each of FIGS. 9-11, one of skill in the art will appreciate that any number of expandable members may be used. Further, although a layer of material is shown in FIGS. 9-11, one of skill in the art will appreciate that any of the layering embodiments of FIGS. 12A-12F may be applied to the embodiments of FIGS. 9-11.

FIG. 12A shows a cross-sectional plan view of at least a portion 1220a of an embodiment of a flow modulating device. FIG. 12A shows a layer of material 1210 defining an aperture 1212 through which an expandable member or expandable material 1224 expands therethrough when inflated. Suture 1214 or other securing element secures the layer of material 1210 and the expandable member (formed by expandable materials 1224) to a frame 1222 (positioned behind layer of material 1210) of a flow modulating device. FIG. 12B is similar to FIG. 12A and includes similarly labeled elements, except that FIG. 12B shows three expandable members or expandable materials 1224a, 1224b, 1224c that each expand, when inflated, through an aperture 1212 defined by a layer of material 1210. The expandable members 1224a, 1224b, 1224c and layer of material 1210 are secured to a frame of a flow modulating device via a coupling element 1214, for example a suture or the like.

FIGS. 12C-12F show cross-sectional views of at least a portion of various embodiments of flow modulating devices. FIGS. 12C-12F show various layerings of materials to secure an expandable member to a frame, and/or various layerings of expandable materials relative to layers of materials to form an expandable member and secure the expandable materials and layers of materials to at least a portion of the frame 1250. For example, the embodiment 1200c of FIG. 12C shows a first layer of material 1234 coupled to a frame 1250. An expandable member 1240 is coupled to the first layer of material 1234 (and thus frame 1250) and underneath a second layer of material 1232. The second layer of material 1232 further secures or couples the expandable member 1240 to the first layer of material 1234 and/or frame 1250. The second layer of material defines an aperture 1230 through which the expandable member 1240 extends or expands when it is at least partially inflated with inflation fluid.

The embodiment 1200d of FIG. 12D is similar to that described in connection with FIGS. 9-11, where a layer of material 1236 is sandwiched between two expandable layers 1260, 1262. The layer of material 1236 secures the two expandable layers 1260, 1262 to at least a portion of the frame 1250 and/or to each other. The layer of material 1236 defines an aperture 1264, such that a pocket is formed between the two expandable layers 1260, 1262. The pocket is the expandable member (bounded by the two expandable layers 1260, 1262) that defines a volume that can be filled with inflation fluid to a plurality of inflation states. For example, the layer of material 1236 may be an intermediate woven, biomaterial, textile, etc. layer between the first and second expandable layers 1260, 1262.

FIG. 12E shows another embodiment 1200e showing a coupling between an expandable member and a frame 1250. An expandable member 1242 is layered on an internal surface of the frame 1250. A layer of material 1238, defining an aperture 1246, secures the expandable member 1242 to at least a portion of the frame 1250. Said another way, the expandable member 1242 is coupled to the frame 1250 beneath the layer of material 1238, such that the expandable member 1242 reversibly expands through the aperture 1246 defined by the layer of material 1238.

FIG. 12F shows another embodiment 1200f illustrating a coupling between an expandable member and a frame 1250. At least a portion of an internal surface of the frame 1250 is coupled to a layer of material 1232. The expandable member 1244 is coupled to the layer of material 1232. The layer of material 1232 forms a base or provides structural elements through which the expandable member 1244 can be secured to the frame 1250.

### Methods

FIG. 13 is a schematic of an embodiment of a method 1300 of modulating flow in a vessel using any of the flow modulating devices described herein. The method 1300 includes receiving a blood pressure of a patient having the flow modulating device implanted therein at block S1310; determining a volume of the inflation fluid to be output from the reservoir into the volume of the one or more expandable members based on the blood pressure at block S1320; and outputting a control signal to the pump to actuate the inflation fluid from the reservoir into the inflation line and into the volume of the one or more expandable members at block S1330. The method 1300 functions to modulate a volume of inflation fluid leaving the reservoir and entering one or more expandable members to modulate a blood flow path through a lumen of the flow modulating device in which the expandable members are installed.

For example, in some embodiments, the blood pressure is in a first blood pressure range, such that the control signal causes the one or more expandable members to be substantially filled with inflation fluid to restrict blood flow through the flow modulating device at optional block S1340. For example, the first blood pressure range may be about 10 mmHg to about 25 mmHg; about 10 mmHg to about 12 mmHg; about 12 mmHg to about 14 mmHg; about 14 mmHg to about 16 mmHg; about 16 mmHg to about 18 mmHg about 18 mmHg to about 20 mmHg; about 20 mmHg to about 22 mmHg; about 22 mmHg to about 25 mmHg.

In some variations, the blood pressure is in a second blood pressure range, such that the control signal causes the one or more expandable members to be partially filled with inflation fluid to at least partially restrict blood flow through the flow modulating device at optional block S1350. For example, the second blood pressure range can be about 25 mmHg to about 35 mmHg; about 25 mmHg to about 27 mmHg; about 27 mmHg to about 29 mmHg; about 29 mmHg to about 31 mmHg; about 31 mmHg to about 33 mmHg; about 33 mmHg to about 35 mmHg.

In some embodiments, the blood pressure is in an exertion-related blood pressure range. At exertion-related blood pressures, the control signal causes no or substantially no inflation fluid to be released from the reservoir (or pumped from the reservoir) into the one or more expandable members so that blood flow is substantially unrestricted through the flow modulating device at optional block S1360. For example, the exertion-related blood pressure range may be greater than about 25 mmHg or greater than about 35 mmHg.

In some aspects of block S1310 of method 1300, a processor, communicatively coupled to the inflation apparatus, receives a blood pressure of a patient having the flow modulating device implanted therein. For example, the blood pressure may be sensed by a sensor that is coupled to the flow modulating device or otherwise in proximity to the flow modulating device. The sensor signal is transmitted to the processor and received by the processor. The processor may also receive an input indicative of the volume of the one or more expandable members. The input, indicative of the volume, may be stored in local memory or transmitted to the processor from a local or remote computing device. The volume of the one or more expandable members may be based on manufacturer specifications, predefined, or otherwise determined (e.g., based on a patient blood pressure).

In some aspects of block S1320 of method 1300, the processor, communicatively coupled to the inflation apparatus, determines a volume of the inflation fluid to be output from the reservoir into the volume of the one or more expandable members based on the blood pressure. The determined volume may be dependent on an inflation diameter of the one or more expandable members, a material property of the one or more expandable members, a filling sequence of the one or more expandable members, or other properties of the one or more expandable members.

In some aspects of block S1320 of method 1300, the control signal causes the one or more expandable members to be inflated to an inflation state of the plurality of inflation states based on the blood pressure.

In some variations, the processor may further determine a pressure of the inflation fluid to be output from the reservoir into the volume of the one or more expandable members, for example, such that the pressure in the one or more expandable members may at least be equal to or greater than a blood pressure. The processor may output a second control signal to the pump to actuate the inflation fluid at the determined pressure from the reservoir into the inflation line and into the volume of the one or more expandable members.

In some embodiments, a processor, communicatively coupled to the inflation apparatus: receives a blood pressure of a patient having the flow modulating device implanted therein; receives a predefined cross-sectional area reduction of a total cross-sectional area of the lumen of the frame; determines an inflation volume per expandable member of the one or more expandable members, based on the blood pressure, to achieve the predefined cross-sectional area reduction; and outputs a control signal to the pump to actuate the determined inflation volume of the inflation fluid from the reservoir into the inflation line and into the volume of the one or more expandable members. The predefined cross-sectional area reduction determines how much of the lumen of the flow modulating device is occluded, and thus how much blood can flow therethrough. For example, a high threshold cross-sectional area reduction can result in more lumen occlusion, while a low threshold cross-sectional area reduction can result in less lumen occlusion and more blood flow. The determined inflation volume to achieve the predefined cross-sectional area reduction may be dependent on an inflation diameter of the one or more expandable members, a material property of the one or more expandable members, a filling sequence of the one or more expandable members, or other properties of the one or more expandable members.

### Example Implantation of Flow Modulating Devices

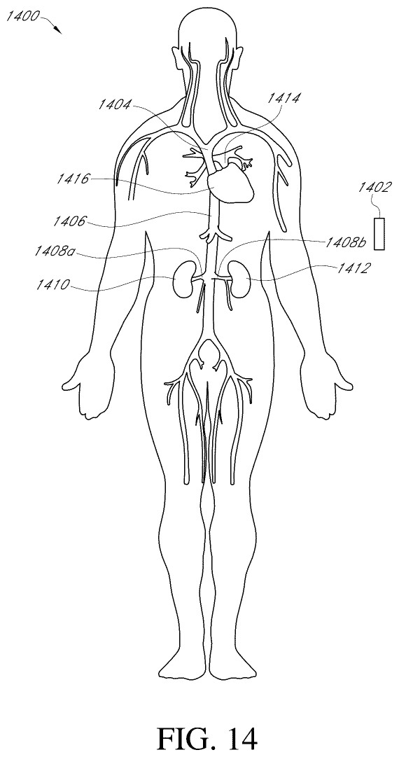

FIG. 14 illustrates a schematic representation of portions of a subject 1400. The flow modulating devices described herein (represented in FIGS. 2A-7B and 9-12F by device 1402) may be introduced (e.g., implanted) in vasculature of the body. In general, the device 1402 may represent any of the flow modulating devices described herein or portions thereof (e.g., devices 20, 120, 220a, 220b, 220c, 320a 320b, 320c, 420a, 420b, 520, 540, 560, 620, 720, 740, 820a, 820b, 920, 1020, 1120, 1200a, 1200b, 1200c, 1200d, 1200e, 1200f, etc.) and may include the same or similar functionality and/or structures. In some examples, the device 1402 may be implanted in or near to a portion of the Superior Vena Cava (SVC) 1404. In some examples, the device 1402 may be implanted in or near to a portion of the Inferior Vena Cava (IVC) 1406. The subject 1400 is illustrated with a representation of a portion of the vasculature system to generally illustrate the SVC 1404 and the IVC 1406 within the subject 1400. However, it is to be understood that no dimensions or relative sizes of components may be inferred from the relative sizes and dimensions of elements in the figures.

The subject 1400 includes a number of vessels and organs that may circulate blood throughout the body. For example, renal veins 1408a and 1408b drain blood from respective right kidney 1410 and left kidney 1412. Renal veins 1408a and 1408b connect to the IVC 1406. Blood from the aorta 1414 flows to the IVC 1406. Blood travels from the aorta 1414 to the abdominal organs including the stomach (not shown), liver (not shown), spleen (not shown), pancreas (not shown), large intestines (not shown), and small intestine (not shown). Following processing of the blood by the liver, blood collects in the central vein. Blood from these central veins converges in the hepatic veins (not shown) which exit the liver and empty into the IVC 1406 to be distributed to the rest of the body.

Portions of the above-recited blood circulating vessels and/or organs may be involved in splanchnic venous circulation that includes blood flow originating from the celiac, superior mesenteric, and inferior mesenteric arteries to the abdominal organs. The splanchnic venous circulation may act as a blood reservoir that can support the need for increased stressed blood volume during periods of elevated sympathetic tone, such as during exertion, to support increased cardiac output and vasodilation of peripheral vessels supporting active muscles.

However, heart failure patients can have multiple comorbidities that prevent the use of this additional blood reservoir. Example comorbidities can include chronic kidney disease, chronotropic incompetence, inability to increase stroke volume, and/or peripheral microvascular dysfunction. This can lead to venous congestion and/or abrupt rises in central venous pressure, pulmonary artery pressure, and/or pulmonary capillary wedge pressure. To alleviate such pressures, the blood reserves within the blood reservoir described above can be used to support the need for increased stressed blood volume during periods of elevated sympathetic tone. The flow modulating devices described herein may be used to ensure that such blood reserves within the blood reservoir can be utilized.

For example, because blood flow from the splanchnic venous circulation is directed through hepatic veins and into the IVC 1406, devices (as described herein) may be placed into the IVC 1406 to limit blood flow to allow the reservoir to expand with increased blood volume. Similarly, devices (as described herein) may be placed into the SVC 1408 to limit blood flow to allow the reservoir to expand with increased blood volume. Furthermore, the flow modulating devices described herein may be placed in either the IVC 1406 and/or SVC 1408 to alleviate pressure in the right side of the atrium of the heart 1416.

In some examples, the flow modulating device 1402 (representing the devices described herein) may be used as a method of treatment to treat any combination of heart failure, chronic kidney disease, chronotropic incompetence, inability to increase stroke volume, and/or peripheral microvascular dysfunction. In addition, the flow modulating device 1402 may be used as a method of treatment to regulate pressure in the right atrium of the heart. Further, the flow modulating device 1402 may be used as a method of treatment to improve function of the kidneys in patients having reduced kidney function due to pressure in the venous system.

As used herein, the term “active” with respect to blood flow management may represent operations carried out by the devices described herein using power or controller induced movement. For example, moving inflation fluid into and out of one or more expandable members of the devices described herein may include the use of battery power, wall outlet power, magnetic field induction, electromagnetic field induction, a positive-displacement pump, an impulse pump, a velocity pump, a steam pump, a valveless pump, and/or other pump using electrical or inductive power.

In some examples, an active control mechanism may include a microcontroller and/or a power source implanted with or integrated with the flow management device. Alternatively, or additionally, an active control mechanism can include a microcontroller and/or a power source in a remote control device, external to the body, or in an implanted remote device (e.g., subcutaneously, intravascularly, etc.), for example. The remote control device may be in wireless communication with the implanted device or connected to the implanted device through one or more leads.

In any of the embodiments described herein, an active mechanism may include a pump fluidly connected to a reservoir; and optionally a manifold fluidly connected to the pump and the reservoir. The pump may be actuated to cause fluid to evacuate the reservoir, possibly through the manifold, and into an inflation line. The inflation is fluidly connected to a volume defined by an expandable member through an inflation port of the expandable member.

As used herein, the term “passive” with respect to blood flow management may represent operations carried out by the devices described herein using passively induced movement. For example, passively causing inflation fluid to evacuate the reservoir and enter an inflation line fluidly connected to a volume defined by an expandable member may include the use of manual pumps or pistons, anatomy responses (e.g., changes in vessel inner diameter, intra-vessel pressure, etc.), blood movement, or the like.

In some examples described herein, the flow modulating devices are operated in more than one mode of operation. For example, one or more expandable members can be inflated to a first inflation state (e.g., used to restrict blood flow through a blood vessel) in a first mode in response to an increase in blood pressure within a first blood pressure range, and inflated to a second inflation state in a second mode in response to an increase in blood pressure within a second blood pressure range that is different (e.g., higher) than the first blood pressure range. The first mode can be that the one or more expandable members are substantially inflated to substantially occlude the lumen of the frame to further restrict blood flow within the blood vessel, and the second mode can be that the one or more expandable members are substantially uninflated or partially inflated to substantially not occlude or partially occlude the lumen of the frame, respectively, to increase the blood flow within the blood vessel.

Any of the implantable or flow modulating devices described herein may be coated with a polymer (e.g., silicones, poly(urethanes), poly(acrylates), or copolymers such as poly(ethylene vinyl acetate), a drug (e.g., heparin, pro-endothelialization drugs, anti-thrombogenic drug, etc.), a textile (e.g., woven, knitted, nonwoven, or braided), tissue (e.g., bovine pericardium, equine pericardium, porcine vena cava, etc.), or a combination thereof. Woven and knitted fabrics may be made from poly (ethylene terephthalate), while the nonwoven fabrics may be made from expanded poly(tetrafluoroethylene). Some textiles may also or alternatively include silk or silk-based materials.

Further, any of the layers of materials described herein may include silk, silk-based materials, nylon, synthetic polymer materials (e.g., silicone, polydioxanone, polyglycolic acid, polyglyconate, polylactic acid, etc.), natural materials (e.g., purified catgut, collagen, sheep intestines, cow intestines, etc.), metal (e.g., Nitinol®, palladium, gold and their alloys, etc.), or a combination thereof.

The flow modulating devices described herein may be part of (or installed within) a stent. The stent may represent a frame or outer frame that provides a support structure for the flow modulating devices when the stent is implanted into a blood vessel. The frame/outer frame may be a self-expanding frame or a balloon-expandable frame. In general, any type of stent may be used with the flow modulating devices. Example stents may include, but are not limited to, bare metal stents, coated stents, drug-eluting stents, biodegradable stents, balloon expandable stents, and self-expandable stents.

The stents described herein may be configured to house all or a portion of the flow modulating devices described herein. Such stents may include an assembly with strut members interconnected by joints that form a series of linked mechanisms that result in a hollow, substantially tube-shaped or oval-shaped element. The stents may be positioned and/or repositioned within a blood vessel to introduce or remove flow modulating devices or device members including, but not limited to, valving, control elements, balloons, flexible members, rigid members, adjustment mechanisms, sensors, coils, wires, and/or magnets. One or more of such device members may be actuated to modify stent shape (or device member shape) for purposes of modifying a flow of fluid through the vessel associated with the implanted stent. Moreover, the stents described herein may partially or fully surround a flow modulating device. For example, a stent or stent portion may surround a portion of a flow modulating device to ensure the device remains in a specified position in a blood vessel. In some examples, the stent surrounds the flow modulating device entirely. In some examples, the stent surrounds the flow modulating device and further continues beyond one or both ends of the device.

The stents described herein may include an outer frame. The outer frame may have a form and structure that varies. For example, the strut members and/or articulated joints may form a mesh-like structure. The strut members may be interconnected in such a way as to form a shaped pattern of cells. For example, any number of strut members may form a ring of the stent such that the strut members are connected by any number of crowns. Any number of rings may form a body of the stent, and the rings may be connected by any number of bridges. Example cell shapes may include, but are not limited to diamond, square, rectangle, triangle, oval, ganglion, or any combination thereof. In some examples, the cells may be evenly shaped and distributed from a first end of the stent to a second end of the stent. In some examples, the cells may include a number of strut members interconnected in such a way that when the stent expands radially, one or more of the cells become longitudinally shorter. Similarly, when the stent constricts radially, one or more of the cells become longitudinally longer.

Constricting portions of the stents described herein may result in an outer frame woven tighter than other portions of the stent that are not constricted. The constriction may push against one or more portions of the flow modulating devices described herein to narrow a pathway through the frame or outer frame and/or to trigger the flow modulating device to begin or end constriction. Similarly, expanding portions of the stents described herein may result in an outer frame woven looser than other portions of the stent that are not expanded. The expansion may release one or more portions of the flow modulating devices described herein to widen a pathway through the frame or outer frame and/or to trigger the flow modulating device to begin or end constriction.

The flow modulating devices described herein may be introduced to a vessel or tissue site using a delivery system. For example, such delivery systems may be used to position catheter tips and/or catheters in various portions of a target vasculature. A delivery system may include a delivery catheter having a pusherwire or the like disposed therein. The pusherwire may be configured to deploy any of the devices described herein, for example by urging the device out of a distal end of the catheter and either actively expanding the device or allowing the device to passively expand once it is no longer constrained by a lumen of the catheter. Any of the devices described herein may be crimped or otherwise compressed such that a cross-sectional area of the device is sized and/or shaped to be delivered through a lumen of a catheter. In some examples, the crimped or compressed device may be transferred to the delivery system using a transfer sheath, or the like. A delivery system can access the vasculature through an access site, such as a radial artery, brachial artery, internal jugular vein, common femoral vein, subclavian veins, or the like.

For example, in a coronary procedure, a catheter tip and/or catheter may be configured to pass from the right atrium into the coronary sinus. For access to the venous circulation, for example, a catheter tip and/or catheter may be configured to pass from the radial artery into the superior vena cava. Further, for central venous access, a catheter tip and/or catheter may be configured to pass from the femoral vein into the inferior vena cava.

In some examples, the delivery system may include a trocar or other suitable delivery device may be used for implanting devices subcutaneously, for example control devices for controlling activation of any of the flow modulating devices described herein. As described elsewhere herein, various control systems may include an implanted remote device that is configured to transmit control signals to a flow modulating device disposed in the vasculature. The control signals may include signals transmitted wirelessly, through a wired connection (e.g., leads), or via magnetic field induction, electromagnetic field induction, or magnetic polarization.

However, it will be understood that the delivery system can refer or generally apply to positioning of catheter tips and/or catheters from a first body chamber or lumen into a second body chamber or lumen, where the catheter tips and/or catheters may be bent when positioned from the first body chamber or lumen into the second body chamber or lumen. A body chamber or lumen can refer to any one of a number of fluid channels, blood vessels (e.g., superior vena cava, inferior vena cava, renal artery, renal vein, etc.), and/or organ chambers (e.g., heart chambers). Additionally, reference herein to “catheters,” “tubes,” “sheaths,” “steerable sheaths,” and/or “steerable catheters” can refer or apply generally to any type of elongate tubular delivery device including an inner lumen configured to slidably receive instrumentation, such as for positioning within an atrium, coronary sinus, superior vena cava, or inferior vena cava, including for example delivery catheters, cannulas, and/or trocars. It will be understood that other types of medical implant devices and/or procedures can be delivered to the coronary sinus, superior vena cava, inferior vena cava, etc. using a delivery system as described herein, including for example ablation procedures, drug delivery, and/or placement of actuator leads.

Described herein are various example medical implants and/or delivery methods. Some examples described herein may be used in combination and/or may be used independently.

Provided below is a list of examples, each of which may include aspects of any of the other examples disclosed herein. Furthermore, aspects of any example described above may be implemented in any of the numbered examples provided below.

Depending on the example, certain acts, events, or functions of any of the processes or algorithms described herein can be performed in a different sequence, may be added, merged, or left out altogether. Thus, in certain examples, not all described acts or events are necessary for the practice of the processes.

### EXAMPLES

### Example 1

A device for modulating blood flow in a vessel, the device comprising: a frame comprising an inflow end and an outflow end, the frame defining a lumen between the inflow end and the outflow end; and a first expandable member coupled to at least one portion of an internal surface of the frame and comprising a first inflation port, the first expandable member defining a first volume configured to receive an inflation fluid therein through the first inflation port, wherein the first expandable member is configured to be reversibly inflatable to a first plurality of inflation states to partially or fully occlude the lumen.

### Example 2

The device of any one of the preceding examples, but particularly Example 1, further comprising an inflation line and an inflation apparatus, wherein the inflation line is configured to fluidly couple the first volume of the first expandable member to the inflation apparatus through the first inflation port to reversibly inflate the first expandable member.

### Example 3

The device of any one of the preceding examples, but particularly Example 1, wherein the first expandable member comprises an inflatable balloon.

### Example 4

The device of any one of the preceding examples, but particularly Example 2, further comprising a second expandable member coupled to at least a second portion of the internal surface of the frame and comprising a second inflation port, wherein the second expandable member defines a second volume configured to receive the inflation fluid therein through the second inflation port.

### Example 5

The device of any one of the preceding examples, but particularly Example 4, wherein: the inflation line is fluidly coupled to the second volume of the second expandable member through the second inflation port; and the second expandable member is reversibly inflatable between a second plurality of inflation states to partially or fully occlude the lumen of the frame.

### Example 6

The device of any one of the preceding examples, but particularly Example 4, wherein the first expandable member is circumferentially offset from the second expandable member along the internal surface of the frame.

### Example 7

The device of any one of the preceding examples, but particularly Example 6, wherein the circumferential offset is between about 60 degrees and about 200 degrees.

### Example 8

The device of any one of the preceding examples, but particularly Example 4, further comprising a third expandable member coupled to at least a third portion of the internal surface of the frame and comprising a third inflation port, wherein the third expandable member defines a third volume configured to receive the inflation fluid therein through the third inflation port.

### Example 9

The device of any one of the preceding examples, but particularly Example 8, wherein: the inflation line is fluidly coupled to the third volume of the third expandable member through the third inflation port; and the third expandable member is reversibly inflatable between a third plurality of inflation states to partially or fully occlude the lumen of the frame.

### Example 10

The device of any one of the preceding examples, but particularly Example 9, wherein the inflation line is directly fluidly connected to the first inflation port, the second inflation port, and the third inflation port, such that the first expandable member, the second expandable member, and the third expandable member are configured to be inflated substantially concurrently.

### Example 11

The device of any one of the preceding examples, but particularly Example 9, wherein the inflation line is directly fluidly connected to the first inflation port and indirectly fluidly connected to the second inflation port and the third inflation port, such that the first expandable member, the second expandable member, and the third expandable member are configured to be inflated substantially sequentially.

### Example 12

The device of any one of the preceding examples, but particularly Example 8, wherein the third expandable member is circumferentially offset from the first expandable member and the second expandable member along the internal surface of the frame.

### Example 13

The device of any one of the preceding examples, but particularly Example 12, wherein the circumferential offset is about 60 degrees to about 150 degrees.

### Example 14

The device of any one of the preceding examples, but particularly Example 1, wherein the first expandable member is coupled to at least the one portion of the internal surface of the frame adjacent to the inflow end of the frame.

### Example 15

The device of any one of the preceding examples, but particularly Example 1, wherein the first expandable member is coupled to at least the one portion of the internal surface of the frame adjacent to the outflow end of the frame.

### Example 16

The device of any one of the preceding examples, but particularly Example 1, wherein the first expandable member is coupled to at least the one portion of the internal surface of the frame substantially equidistant between the inflow end and the outflow end of the frame.

### Example 17

The device of any one of the preceding examples, but particularly Example 16, wherein the first expandable member is a cylindrical shape such that the first expandable member extends between the inflow end and the outflow end of the frame.

### Example 18

The device of any one of the preceding examples, but particularly Example 1, wherein the coupling of the first expandable member to at least the one portion of the internal surface of the frame is such that the first expandable member extends at least partially along a circumference of the internal surface of the frame.

### Example 19

The device of any one of the preceding examples, but particularly Example 18, wherein the first expandable member extends about 90 degrees to about 210 degrees of the circumference of at least the one portion of the internal surface of the frame.

### Example 20

The device of any one of the preceding examples, but particularly Example 1, wherein the first expandable member comprises an embedded radiopaque marker.

### Example 21

The device of any one of the preceding examples, but particularly Example 1, wherein: the internal surface of the frame comprises a first layer of material; and the first expandable member is coupled to the first layer of material.

### Example 22

The device of any one of the preceding examples, but particularly Example 1, wherein: the internal surface of the frame comprises a first layer of material defining an aperture; and the first expandable member is coupled to the frame beneath the first layer of material and configured to reversibly expand through the aperture defined by the first layer of material.

### Example 23

The device of any one of the preceding examples, but particularly Example 21, further comprising a second layer of material disposed over the first expandable member and coupled to the first layer of material, wherein the second layer of material defines an aperture through which the first expandable member expands when inflated to one of the first plurality of inflation states.

### Example 24

The device of any one of the preceding examples, but particularly Example 1, further comprising a first layer of material defining an aperture, wherein: the first expandable member is formed of a first expandable layer and a second expandable layer, the first layer of material defining the aperture is positioned between the first expandable layer and the second expandable layer, such that the first expandable layer and the second expandable layer together define the first volume of the first expandable member at the aperture of the first layer of material, and the first layer of material is configured to secure the first and second expandable layers to at least the one portion of the internal surface of the frame.

### Example 25

The device of any one of the preceding examples, but particularly Example 24, wherein the first and second expandable layers comprise or are formed of a thermoplastic elastomer.

### Example 26

The device of any one of the preceding examples, but particularly Example 24, wherein the first layer of material comprises or is formed of one or more of: a polymer, a biomaterial, or a textile.

### Example 27

The device of any one of the preceding examples, but particularly Example 1, wherein the first expandable member comprises a first end opposite a second end about a cross-sectional axis of the first expandable member, wherein the first end is substantially convex, when fully inflated, and the second end is substantially convex, when fully inflated.

### Example 28

The device of any one of the preceding examples, but particularly Example 1, wherein the first expandable member comprises a first end opposite a second end about a cross-sectional axis of the first expandable member, wherein the first end is substantially convex, when fully inflated, and the second end is substantially planar, when fully inflated.

### Example 29

The device of any one of the preceding examples, but particularly Example 1, wherein the expandable member comprises a first expandable layer and a second expandable layer with an intermediate woven layer between the first and second expandable layers.

### Example 30

The device of any one of the preceding examples, but particularly Example 29, wherein the intermediate woven layer defines an aperture so that the first expandable layer and the second expandable layer together form the first expandable member at the aperture.

### Example 31

The device of any one of the preceding examples, but particularly Example 1, wherein the first expandable member comprises a compliant material.

### Example 32

The device of any one of the preceding examples, but particularly Example 31, wherein the compliant material comprises a polyurethane, a silicone, or a combination thereof.

### Example 33

The device of any one of the preceding examples, but particularly Example 1, wherein the first expandable member comprises a non-compliant material.

### Example 34

The device of any one of the preceding examples, but particularly Example 33, wherein the non-compliant material comprises a polyester, a nylon, or a combination thereof.

### Example 35

The device of any one of the preceding examples, but particularly Example 1, wherein the frame comprises a stent.

### Example 36

The device of any one of the preceding examples, but particularly Example 1, wherein the vessel is a superior vena cava or an inferior vena cava.

### Example 37

The device of any one of the preceding examples, but particularly Example 1, wherein an outer surface of the first expandable member and at least a second portion of the inner surface of the frame together define a stasis reduction flow path.

### Example 38

The device of any one of the preceding examples, but particularly Example 37, wherein the stasis reduction flow path extends along at least a portion of a longitudinal length of the frame to fluidly connect the inflow end to the outflow end.

### Example 39

The device of any one of the preceding examples, but particularly Example 37, wherein the stasis reduction flow path comprises one or more longitudinally extending conduits coupled to at least the second portion of the inner surface of the frame.

### Example 40

A method of modulating blood flow within a blood vessel, comprising using the device of any one of Examples 1-39 to restrict blood flow within the blood vessel.

### Example 41

A method of treatment for a subject having one or both of: congestive heart failure or chronic kidney disease, comprising using the device of any one of Examples 1-39 to modulate blood flow within a blood vessel of the subject.

### Example 42

A system for modulating blood flow through a vessel, the system comprising: an inflation apparatus comprising a pump and a reservoir configured to contain an inflation fluid; a flow modulating device comprising: a frame comprising an inflow end and an outflow end, the frame defining a lumen comprising an internal surface between the inflow end and the outflow end, and one or more expandable members coupled to at least one portion of the internal surface of the frame and comprising an inflation port, the one or more expandable members each defining a volume configured to receive an inflation fluid therein through the inflation port; and an inflation line configured to fluidly connect the reservoir of the inflation apparatus to the volume of each of the one or more expandable members through each inflation port, wherein the one or more expandable members are each reversibly inflatable to a plurality of inflation states to partially or fully occlude the lumen of the frame.

### Example 43

The system of any one of the preceding examples, but particularly Example 42, wherein the inflation apparatus is configured to deliver the inflation fluid to the volume of each of the one or more expandable members such that a pressure in the volume is at least about 10 mmHg.

### Example 44

The system of any one of the preceding examples, but particularly Example 42, wherein the inflation apparatus is configured to deliver the inflation fluid to the volume of each of the one or more expandable members such that a pressure in the volume is between about 10 mmHg and about 40 mmHg.

### Example 45

The system of any one of the preceding examples, but particularly Example 43, wherein the inflation fluid comprises one or more of: a gas, saline, heparinized saline, blood, contrast, or a combination thereof.

### Example 46

The system of any one of the preceding examples, but particularly Example 42, further comprising a processor communicatively coupled to the inflation apparatus and configured to: receive a blood pressure of a patient having the flow modulating device implanted therein, receive an input indicative of the volume of the one or more expandable members, determine a volume of the inflation fluid to be output from the reservoir into the volume of the one or more expandable members based on the blood pressure, and output a control signal to the pump to actuate the inflation fluid from the reservoir into the inflation line and into the volume of the one or more expandable members, wherein the one or more expandable members are inflated to a first inflation state of the plurality of inflation states based on the blood pressure.

### Example 47

The system of any one of the preceding examples, but particularly Example 46, wherein the processor is further configured to: determine a pressure of the inflation fluid to be output from the reservoir into the volume of the one or more expandable members, based on the blood pressure, and output a second control signal to the pump to actuate the inflation fluid at the determined pressure from the reservoir into the inflation line and into the volume of the one or more expandable members.

### Example 48

The system of any one of the preceding examples, but particularly Example 46, wherein the determined pressure of the inflation fluid in the one or more expandable members is higher than the blood pressure of the patient.

### Example 49

The system of any one of the preceding examples, but particularly Example 46, further comprising a sensor communicatively coupled to the processor and configured to measure the blood pressure.

### Example 50

The system of any one of the preceding examples, but particularly Example 46, further comprising an antenna communicatively coupled to the processor and configured to receive the blood pressure from a computing device.

### Example 51

The system of any one of the preceding examples, but particularly Example 46, wherein the blood pressure is in a first blood pressure range, such that the first inflation state is configured to restrict blood flow through the lumen of the frame.

### Example 52

The system of any one of the preceding examples, but particularly Example 51, wherein the first blood pressure range is about 10 mmHg to about 25 mmHg.

### Example 53

The system of any one of the preceding examples, but particularly Example 46, wherein the blood pressure is in a second blood pressure range, such that the first inflation state is configured to partially restrict blood flow through the lumen of the frame.

### Example 54

The system of any one of the preceding examples, but particularly Example 53, wherein the second blood pressure range is about 25 mmHg to about 35 mmHg.

### Example 55

The system of any one of the preceding examples, but particularly Example 46, wherein the blood pressure is in an exertion-related blood pressure range, such that the first inflation state is configured to substantially unrestrict blood flow through the lumen of the frame.

### Example 56

The system of any one of the preceding examples, but particularly Example 55, wherein the exertion-related blood pressure range is greater than about 25 mmHg or greater than about 35 mmHg.

### Example 57

The system of any one of the preceding examples, but particularly Example 42, further comprising a processor communicatively coupled to the inflation apparatus and configured to: receive a blood pressure of a patient having the flow modulating device implanted therein, receive a predefined cross-sectional area reduction of a total cross-sectional area of the lumen of the frame, determine an inflation volume per expandable member of the one or more expandable members, based on the blood pressure, to achieve the predefined cross-sectional area reduction, and output a control signal to the pump to actuate the determined inflation volume of the inflation fluid from the reservoir into the inflation line and into the volume of the one or more expandable members, wherein the one or more expandable members are inflated to a first inflation state of the plurality of inflation states based on the blood pressure.

### Example 58

The system of any one of the preceding examples, but particularly Example 42, wherein the inflation apparatus is positioned subcutaneously in a patient; and the flow modulating device is implanted intraluminally.

### Example 59

The system of any one of the preceding examples, but particularly Example 42, wherein the inflation apparatus is externally coupled to a patient; and the flow modulating device is implanted intraluminally.

### Example 60

The system of any one of the preceding examples, but particularly Example 42, wherein the inflation apparatus is implanted in a patient; and the flow modulating device is implanted intraluminally.

### Example 61

The system of any one of the preceding examples, but particularly Example 42, wherein the frame comprises a stent.

### Example 62

The system of any one of the preceding examples, but particularly Example 42, wherein the vessel is a superior vena cava or an inferior vena cava.

### Example 63

A method of modulating blood flow within a blood vessel, comprising using the system of any one of Examples 42-62 to modulate blood flow within the blood vessel.

### Example 64

A method of treatment for a subject having one or both of: congestive heart failure or chronic kidney disease, comprising using the system of any one of Examples 42-62 to modulate blood flow within a blood vessel of the subject.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It should be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

The systems and methods of the embodiments and variations described herein can be embodied and/or implemented at least in part as a machine configured to receive a computer-readable medium storing computer-readable instructions. The instructions may be executed by computer-executable components integrated or in communication with the system and one or more portions of the processor on or in communication with the flow modulating device and/or computing device. The computer-readable medium can be stored on any suitable computer-readable media such as RAMs, ROMs, flash memory, EEPROMs, optical devices (e.g., CD or DVD), hard drives, floppy drives, or any suitable device. The computer-executable component is preferably a general or application-specific processor, but any suitable dedicated hardware or hardware/firmware combination can alternatively or additionally execute the instructions.

As used in the description and claims, the singular form “a”, “an” and “the” include both singular and plural references unless the context clearly dictates otherwise. For example, the term “expandable member” may include, and is contemplated to include, a plurality of expandable members. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A device for modulating blood flow in a vessel, the device comprising:
a braided tubular frame having an inflow end and an outflow end, the tubular frame defining a lumen between the inflow end and the outflow end, the tubular frame sized and shaped for deployment in a vessel; and
a first expandable member coupled to an internal surface of the tubular frame and comprising a first inflation port;
wherein the first expandable member is inflatable for partially occluding the lumen of the tubular frame, thereby allowing selective modulation of blood flow through the vessel.

2. The device of claim 1, further comprising an inflation line and a pump, wherein the inflation line is configured to fluidly couple the first expandable member to the pump through the first inflation port.

3. The device of claim 1, wherein the first expandable member is an inflatable balloon.

4. The device of claim 1, further comprising a second expandable member coupled to the internal surface of the tubular frame, the second expandable member comprising a second inflation port.

5. The device of claim 4, wherein the first expandable member is circumferentially offset from the second expandable member in the range of 60 to 200 degrees.

6. The device of claim 1, wherein the first expandable member is coupled to the internal surface of the tubular frame adjacent the inflow end of the tubular frame.

7. The device of claim 1, wherein the first expandable member is coupled to the internal surface of the tubular frame substantially equidistant between the inflow end and the outflow end of the tubular frame.

8. The device of claim 1, wherein the first expandable member has a cylindrical shape that extends between the inflow end and the outflow end of the tubular frame.

9. The device of claim 1, further comprising a layer of material positioned within the lumen of the tubular frame.

10. The device of claim 9, wherein the first expandable member is positioned between the interior surface of the tubular frame and the layer of material.

11. The device of claim 10, wherein the layer of material includes an opening for allowing the first expandable member to extend therethrough.

12. The device of claim 1, wherein the first expandable member comprises a compliant material formed with a material comprising polyurethane, a silicone, or a combination thereof.

13. The device of claim 1, wherein the tubular frame is sized for placement in a superior vena cava or an inferior vena cava.

14. A device for modulating blood flow in a vessel, the device comprising:
an elongate tubular frame having an axial lumen extending between an inflow end and an outflow end;
an expandable member coupled to an internal surface of the frame;
an inflation port positioned on the expandable member for receiving an inflation fluid;
a blood pressure sensor for determining blood pressure adjacent the expandable member; and
a motorized pump for altering a volume of the expandable member via transfer of inflation fluid to and from the expandable member, the motorized pump being controlled by feedback from the sensor.

15. The device of claim 14, further comprising a reservoir, wherein the motorized pump transfers the inflation fluid between the reservoir and the expandable member through an inflation line.

16. The device of claim 15, wherein the inflation fluid is saline.

17. The device of claim 16, wherein the frame is a braided stent.

18. An implantable device for modulating the flow of blood through a blood vessel, the device comprising:
a collapsible elongate tubular stent having a lumen extending therethrough;
an adjustable occluding member disposed within the lumen of the frame;
a blood pressure sensor for determining blood pressure adjacent the occluding member; and
a motor for adjusting a dimension of the occluding member, the motor being controlled by feedback from the sensor.

19. The implantable device of claim 18, wherein the implantable device is sized for placement within an inferior vena cava and the occluding member is adjustable for modulating the flow of blood through the lumen of the stent for alleviating symptoms caused by congestive heart failure.

20. The implantable device of claim 19, wherein the occluding member is an expandable member and wherein the expandable member is fillable with a saline via an inflation line coupled to a pump.
